(19) United States
(12) Patent Application Publication
(10) Pub. No.: US 2011/0016541 A1
(43) Pub. Date: Jan. 20, 2011

(54) GENOME EDITING OF SENSORY-RELATED GENES IN ANIMALS

(75) Inventors: Edward Weinstein, St. Louis, MO (US); Xiaoxia Cui, St. Louis, MO (US); Phil Simmons, St. Louis, MO (US)

Correspondence Address: POLSINELLI SHUGHART PC, 700 W. 47TH STREET, SUITE 1000, KANSAS CITY, MO, 64112-1802, US

(73) Assignee: SIGMA-ALDRICH CO., St. Louis, MO (US)

(21) Appl. No.: 12/842,719

(22) Filed: Jul. 23, 2010

Related U.S. Application Data
(63) Continuation-in-part of application No. 12/592,852, filed on Dec. 3, 2009.
(60) Provisional application No. 61/343,287, filed on Apr. 26, 2010.
(60) Provisional application No. 61/323,702, filed on Apr. 13, 2010.
(60) Provisional application No. 61/323,719, filed on Apr. 13, 2010.
(60) Provisional application No. 61/323,698, filed on Apr. 13, 2010.
(60) Provisional application No. 61/309,729, filed on Mar. 2, 2010.
(60) Provisional application No. 61/308,089, filed on Feb. 25, 2010.
(60) Provisional application No. 61/336,000, filed on Jan. 14, 2010.
(60) Provisional application No. 61/263,904, filed on Nov. 24, 2009.
(60) Provisional application No. 61/263,696, filed on Nov. 23, 2009.
(60) Provisional application No. 61/245,877, filed on Sep. 25, 2009.
(60) Provisional application No. 61/232,620, filed on Aug. 10, 2009.
(60) Provisional application No. 61/228,419, filed on Jul. 24, 2009.
(60) Provisional application No. 61/200,985, filed on Dec. 4, 2008.
(60) Provisional application No. 61/205,970, filed on Jan. 26, 2009.

(51) Int. Cl. A01K 67/027; A01K 67/00; C12N 5/07; G01N 33/00

(52) U.S. Cl. 800/3; 800/13; 800/15; 800/14; 800/17; 435/325; 435/350; 435/351; 435/366; 435/363; 435/352; 435/353

(57) ABSTRACT

The present invention provides genetically modified animals and cells comprising edited chromosomal sequences encoding proteins that are associated with nociception or taste disorders. In particular, the animals or cells are generated using a zinc finger nuclease-mediated editing process. Also provided are methods of using the genetically modified animals or cells disclosed herein to screen agents for toxicity and other effects.

### CROSS-REFERENCE TO RELATED APPLICATIONS

This application claims the priority of U.S provisional application No. 61/343,287, filed Apr. 26, 2010, U.S. provisional application No. 61/323,702, filed Apr. 13, 2010, U.S. provisional application No. 61/323,719, filed Apr. 13, 2010, U.S. provisional application No. 61/323,698, filed Apr. 13, 2010, U.S. provisional application No. 61/309,729, filed Mar. 2, 2010, U.S. provisional application No. 61/308,089, filed Feb. 25, 2010, U.S. provisional application No. 61/336,000, filed Jan. 14, 2010, U.S. provisional application No. 61/263,904, filed Nov. 24, 2009, U.S. provisional application No. 61/263,696, filed Nov. 23, 2009, U.S. provisional application No. 61/245,877, filed Sep. 25, 2009, U.S. provisional application No. 61/232,620, filed Aug. 10, 2009, U.S. provisional application No. 61/228,419, filed Jul. 24, 2009, and is a continuation in part of U.S. non-provisional application Ser. No. 12/592,852, filed Dec. 3, 2009, which claims priority to U.S. provisional 61/200,985, filed Dec. 4, 2008 and U.S. provisional application 61/205,970, filed Jan. 26, 2009, all of which are hereby incorporated by reference in their entirety.

### FIELD OF THE INVENTION

The invention generally relates to genetically modified animals or cells comprising at least one edited chromosomal sequence encoding a sensory-related protein, including proteins related to the encoding and neural processing related to nociception and taste. In particular, the invention relates to the use of a zinc finger nuclease-mediated process to edit chromosomal sequences encoding sensory-related proteins in animals or cells.

### BACKGROUND OF THE INVENTION

The vast majority of drugs, including potential analgesics, fail to successfully proceed through the mandatory three phases of drug testing to gain approval for use in humans. Most candidate drugs fail due to unforeseen toxicology, or other adverse side effect, that arises in humans during drug testing, despite the absence of such effects found during testing in animal models, typically mice.

One reason for this failure of mouse models to predict adverse side effects in humans is because the mouse and human proteins on which the drugs act are different. Even if the target protein of a drug in a mouse and a human are encoded by genetic homologs, the proteins produced by these homologs are rarely identical, and rarely are these target proteins expressed in the same conditions, quantities, and isoforms found in humans. An additional limitation of existing mouse models, particularly when used to evaluate compounds that modulate sensory functions, such as nociception or taste, is that the available repertoire of behavioral evaluations of mice related to sensory disorders are difficult to interpret, and as such may be poor predictors of responses in humans. As a result, the outcomes of pre-clinical studies using mouse models may not be predictive of the outcome in humans.

Despite the known shortcomings of the mouse model, the selection of alternative animal models is limited in part by the availability of techniques needed to edit a particular target gene associated with sensory disorders. Conventional methods such as gene knockout technology may be used to edit a particular gene in a potential model organism, but the gene knockout technology has been reliably developed in only a limited number of organisms such as mice. Ideally, the selection of organism in which to model a complex sensory disorder should be based on the organism's ability to exhibit the characteristics of the disorder as well as its amenability to existing research methods, rather the organism's amenability to the gene editing techniques necessary to create a suitable model organism.

One advantage of using rat models for disease (compared to mice) is that rat physiology and biochemistry often more faithfully recapitulate the human condition. Additionally, because rats are more intelligent than mice, they can be tested for a wider repertoire of behaviors. As a result, candidate drugs or chemicals can be screened for previously unforeseen effects on physiology, learning, memory, depression, anxiety, addiction, and sensory functions.

A need exists for animals with modification to one or more genes associated with human sensory disorders to be used as model organisms in which to study these disorders. The genetic modifications may include gene knockouts, expression, modified expression, or over-expression of alleles that either cause or are associated with sensory diseases in humans. Further, a need exists for a means for screening and assessing potential therapeutic drugs using an animal model for characteristics including efficacy and side effects, with actual human proteins involved in the animal model's response to the drug.

### SUMMARY OF THE INVENTION

One aspect of the present disclosure encompasses a genetically modified animal comprising at least one edited chromosomal sequence encoding a sensory-related protein.

A further aspect provides a non-human embryo comprising at least one RNA molecule encoding a zinc finger nuclease that recognizes a chromosomal sequence encoding a sensory-related protein, and, optionally, at least one donor polynucleotide comprising a sequence encoding the sensory-related protein.

Another aspect provides cell comprising at least one edited chromosomal sequence encoding a sensory-related protein.

Yet another aspect encompasses a method for assessing the effect of an agent in an animal. The method comprises administering the agent to a genetically modified animal comprising at least one edited chromosomal sequence encoding a sensory-related protein, obtaining a parameter from the genetically modified animal, and comparing the selected parameter obtained from the genetically modified animal to the selected parameter obtained from a wild-type animal contacted with the same agent. The selected parameter is chosen from (a) rate of elimination of the agent or at least one agent metabolite; (b) circulatory levels of the agent or at least one agent metabolite; (c) bioavailability of the agent or at least one agent metabolite; (d) rate of metabolism of the agent or at least one agent metabolite; (e) rate of clearance of the agent or at least one agent metabolite; (f) toxicity of the agent or at least one agent metabolite; (g) disposition of the agent or the at least one agent metabolite; (h) extrahepatic contribution to the rate of metabolism or the rate of clearance of the agent or the at least one agent metabolite, and (i) ability of the agent to modify an incidence or indication of a sensory disorder in the genetically modified animal. The sensory disorder is chosen from a nociception disorder, a taste disorder, or any combination thereof.

Still yet another aspect encompasses a method for assessing at least one indication of a sensory disorder in an animal model, the method comprising comparing an assay obtained from the animal model to the assay obtained from a wild-type animal. The sensory disorder assessed using this method is chosen from a nociception disorder, a taste disorder, and combinations thereof. The animal model used in this method comprises a genetically modified animal comprising at least one edited chromosomal sequence encoding a sensory-related protein. The assay obtained from the animal model and the wild-type animal is chosen from one or more of: a) a behavioral assay, b) a physiological assay, c) a whole animal assay, d) a tissue assay, e) a cell assay, a taste or odor preference assay, and g) a biomarker assay.

Other aspects and features of the disclosure are described more thoroughly below.

### DETAILED DESCRIPTION OF THE INVENTION

The present disclosure provides a genetically modified animal or animal cell comprising at least one edited chromosomal sequence encoding a sensory-related protein. The edited chromosomal sequence may be (1) inactivated, (2) modified, or (3) comprise an integrated sequence. An inactivated chromosomal sequence is altered such that a functional protein is not made. Thus, a genetically modified animal comprising an inactivated chromosomal sequence may be termed a “knock out” or a “conditional knock out.” Similarly, a genetically modified animal comprising an integrated sequence may be termed a “knock in” or a “conditional knock in.” As detailed below, a knock in animal may be a humanized animal. Furthermore, a genetically modified animal comprising a modified chromosomal sequence may comprise a targeted point mutation(s) or other modification such that an altered protein product is produced. The chromosomal sequence encoding the sensory-related protein generally is edited using a zinc finger nuclease-mediated process. Briefly, the process comprises introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a sensory-related protein using targeted zinc finger nuclease technology is rapid, precise, and highly efficient.

### (I) Genetically Modified Animals

One aspect of the present disclosure provides a genetically modified animal in which at least one chromosomal sequence encoding a sensory-related protein has been edited. For example, the edited chromosomal sequence may be inactivated such that the sequence is not transcribed and/or a functional sensory-related protein is not produced. Alternatively, the edited chromosomal sequence may be modified such that it codes for an altered sensory-related protein. For example, the chromosomal sequence may be modified such that at least one nucleotide is changed and the expressed sensory-related protein comprises at least one changed amino acid residue (missense mutation). The chromosomal sequence may be modified to comprise more than one missense mutation such that more than one amino acid is changed. Additionally, the chromosomal sequence may be modified to have a three nucleotide deletion or insertion such that the expressed sensory-related protein comprises a single amino acid deletion or insertion, provided such a protein is functional. The modified protein may have altered substrate specificity, altered enzyme activity, altered kinetic rates, and so forth. Furthermore, the edited chromosomal sequence may comprise an integrated sequence and/or a sequence encoding an orthologous protein associated with a sensory disorder. The genetically modified animal disclosed herein may be heterozygous for the edited chromosomal sequence encoding a protein associated with a sensory disorder. Alternatively, the genetically modified animal may be homozygous for the edited chromosomal sequence encoding a protein associated with a sensory disorder.

In one embodiment, the genetically modified animal may comprise at least one inactivated chromosomal sequence encoding a sensory-related protein. The inactivated chromosomal sequence may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional sensory-related protein is not produced. Such an animal may be termed a “knockout.” The inactivated chromosomal sequence comprises no exogenously introduced sequence. Also included herein are genetically modified animals in which two, three, four, five, six, seven, eight, nine, or ten or more chromosomal sequences encoding proteins associated with sensory disorders.

In another embodiment, the genetically modified animal may comprise at least one edited chromosomal sequence encoding an orthologous protein associated with a sensory disorder. The edited chromosomal sequence encoding an orthologous sensory-related protein may be modified such that it codes for an altered protein. For example, the edited chromosomal sequence encoding a sensory-related protein may comprise at least one modification such that an altered version of the protein is produced. In some embodiments, the edited chromosomal sequence comprises at least one modification such that the altered version of the sensory-related protein results in a sensory disorder in the animal. In other embodiments, the edited chromosomal sequence encoding a sensory-related protein comprises at least one modification such that the altered version of the protein protects against a sensory disorder in the animal. The modification may be a missense mutation in which substitution of one nucleotide for another nucleotide changes the identity of the coded amino acid.

In yet another embodiment, the genetically modified animal may comprise at least one chromosomally integrated sequence. The chromosomally integrated sequence may encode an orthologous sensory-related protein, an endogenous sensory-related protein, or combinations of both. For example, a sequence encoding an orthologous protein or an endogenous protein may be integrated into a chromosomal sequence encoding a protein such that the chromosomal sequence is inactivated, but wherein the exogenous sequence may be expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a sensory-related protein may be integrated into a “safe harbor” locus, such as the Rosa26 locus, HPRT locus, or AAV locus. In one iteration of the disclosure, an animal comprising a chromosomally integrated sequence encoding a sensory-related protein may be called a “knock-in”, and it should be understood that in such an iteration of the animal, no selectable marker is present. The present disclosure also encompasses genetically modified animals in which two, three, four, five, six, seven, eight, nine, or ten or more sequences encoding protein(s) associated with sensory disorders are integrated into the genome.

The chromosomally integrated sequence encoding a sensory-related protein may encode the wild type form of the protein. Alternatively, the chromosomally integrated sequence encoding a sensory-related protein may comprise at least one modification such that an altered version of the protein is produced. In some embodiments, the chromosomally integrated sequence encoding a sensory-related protein comprises at least one modification such that the altered version of the protein produced causes a sensory disorder. In other embodiments, the chromosomally integrated sequence encoding a sensory-related protein comprises at least one modification such that the altered version of the protein protects against the development of a sensory disorder.

In an additional embodiment, the genetically modified animal may be a “humanized” animal comprising at least one chromosomally integrated sequence encoding a functional human sensory-related protein. The functional human sensory-related protein may have no corresponding ortholog in the genetically modified animal. Alternatively, the wild-type animal from which the genetically modified animal is derived may comprise an ortholog corresponding to the functional human sensory-related protein. In this case, the orthologous sequence in the “humanized” animal is inactivated such that no functional protein is made and the “humanized” animal comprises at least one chromosomally integrated sequence encoding the human sensory-related protein. For example, a humanized animal may comprise an inactivated abat sequence and a chromosomally integrated human ABAT sequence. Those of skill in the art appreciate that “humanized” animals may be generated by crossing a knock out animal with a knock in animal comprising the chromosomally integrated sequence.

In yet another embodiment, the genetically modified animal may comprise at least one edited chromosomal sequence encoding a sensory-related protein such that the expression pattern of the protein is altered. For example, regulatory regions controlling the expression of the protein, such as a promoter or transcription binding site, may be altered such that the sensory-related protein is over-produced, or the tissue-specific or temporal expression of the protein is altered, or a combination thereof. Alternatively, the expression pattern of the sensory-related protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence, such as a chromosomal sequence encoding a sensory-related protein. The genetically modified animal comprising the lox-flanked chromosomal sequence encoding a sensory-related protein may then be crossed with another genetically modified animal expressing Cre recombinase. Progeny animals comprising the lox-flanked chromosomal sequence and the Cre recombinase are then produced, and the lox-flanked chromosomal sequence encoding a sensory-related protein is recombined, leading to deletion or inversion of the chromosomal sequence encoding the protein. Expression of Cre recombinase may be temporally and conditionally regulated to effect temporally and conditionally regulated recombination of the chromosomal sequence encoding a sensory-related protein.

### (a) Sensory-Related Proteins

Sensory-related proteins are a diverse set of proteins associated with the encoding and neural processing associated with sensory processes including but not limited to nociception and taste.

(i) Nociception and Pain

Nociception, also known as nocioception or nociperception, is defined herein as the neural processes involved in encoding and processing noxious stimuli. The noxious stimuli is encoded by nociceptors (also known as pain receptors), defined herein as the dendrites or nerve endings of specialized neurons capable of detecting mechanical, thermal or chemical changes above a threshold stimulation level. The cell bodies of the sensory neurons that include nociceptors are typically located outside the spinal column in a dorsal root ganglion.

Nociceptors are found in many locations throughout the body of an organism including but not limited to the skin, periosteum, muscle, bladder, digestive tract, and joint surfaces, but are typically most concentrated in the skin near the external surface of the organism. Once stimulated, the nociceptor transmits a signal along the lateral spinothalamic tract of the spinal cord to the brain, and may trigger a variety of autonomic responses including but not limited to pallor, diaphoresis, tachycardia, hypertension, lightheadedness, nausea and fainting. In addition, if the nociceptor signals reach consciousness, a sensation of pain may result.

The signals produced by the nociceptors may be modified by a variety of means including but not limited to extracellular mediator compounds which may modify the sensitivity of the nociceptors, nociceptor inhibitory neurons which may inhibit the signals of nociception transmitting neurons, and structures such as the periaqueductal gray of the brain's tectum which may modify the nociception signal before the signal reaches consciousness. The degree of modification of the nociception signals is influenced by the binding of mediator compounds to a variety of receptors such as opioid receptors associated with the nociceptors and associated neural structures.

The peripheral termini of nociceptors detect noxious stimuli and transduce the stimuli into electrical energy. Nociceptors may be classified in terms of the type of noxious stimulus to which the nociceptor is responsive. A thermal nociceptor is activated by noxious heat or cold at various temperatures. Mechanical nociceptors respond to excess pressure or mechanical deformation, including incisions that break the skin's surface. Chemical nociceptors respond to a wide variety of chemical compounds including spices commonly used in cooking such as capsaicin, environmental irritants such as bee toxins or acrolein, a component of cigarette smoke, and certain endogenous ligands and fatty acid amines arising from changes in internal tissues. Silent or sleeping nociceptors are responsive to stimuli only at the onset of inflammation within the surrounding tissue.

Although nociceptors typically generate a signal once a set threshold of stimulation is exceeded, this threshold may be modified as a result of the degree or duration of stimulation by noxious stimuli, or due to damage or malfunction of the nociceptors or associated neurons. For example, the excitation of nociceptor nerve fibers may become greater as the noxious stimulus continues, leading to hyperalgesia. Hypoalgesia results from a reduced excitation of nociceptor fibers after continued stimulation. Allodynia may result from damage to a nociceptor in the peripheral nerves. Analgesia, the complete inhibition of nociceptor signaling while conscious, may result from the inhibition of nociceptor signaling or the reduction of the signal's effect in the central nervous system. Neuralgia, the sensation of pain without stimulation of the nociceptors, may result from a variety of causes such as ion gate malfunctions, ectopic signaling by mechanically sensitive nociceptors, cross signals between sensory nerve fibers, and malfunctions in the central nervous system.

The nociceptors and associated neural structures may experience one of at least several dysfunctions, resulting in a nocioception disorder. Non-limiting examples of a nocioception disorder include hereditary sensory and autonomic neuropathy (HSAN), type 1 (HSAN-1) such as hereditary sensory radicular neuropathy, ulcero-mutilating neuropathy, thevenard syndrome, familial trophoneurosis, mal perforant du pied, familial syringomyelia, and Charcot-Marie-Tooth type 2B syndrome; HSAN-2 such as congenital sensory neuropathy or Morvan's disease; HSAN-3 such as familial dysautonomia (FD) or Riley-Day syndrome; HSAN-4 such as congenital insensitivity to pain with anhidrosis (CIPA); and HSAN-5 such as congenital insensitivity to pain with partial anhidrosis.

(ii) Taste

Taste, as defined herein, is the conscious sensation resulting from the detection of chemical compounds by a plurality of taste receptors. Taste receptors are defined as dendrites or nerve endings that detect a compound and encode this detection as an electrical signal that is processed by the central nervous system, resulting in the sensation of taste. Each type of taste receptor, which includes a protein encoded by a particular gene, is typically responsive to only a narrow class of compounds. A mixture of compounds, each of which stimulates one type of taste receptor, may generate a multitude of signals from different types of taste receptors. In humans, the signals are carried to the brain via three cranial nerves: the facial nerve (VII), the glossopharyngeal nerve (IX) and a branch of the vagus nerve (X). In the brain, the signals are combined to produce a flavor sensation.

Most flavors may be defined as combinations of a finite and small number of “basic tastes” associated with particular types of taste receptors. Non-limiting examples of basic tastes include bitterness, saltiness, sourness, sweetness, and savoriness/umami. Additional “basic tastes” discovered or hypothesized in humans and other species include fattiness, calcium, dryness/astringency, metallicness, prickliness/hotness, coolness, numbness, and heartiness/kokumi. Each of the flavors is associated with a particular type of taste receptor encoded by a particular gene or family of genes.

Taste perception may vary between individual organisms due at least one of several factors, including but not limited to aging of the organism, color/vision impairments, hormonal influences, genetic variations, oral temperature, drugs and other chemicals, natural substances such as Miracle fruit, which temporarily makes sour foods taste sweeter, CNS Tumors and other neurological causes such as temporal lobe lesions and zinc deficiency. The stomach also contains receptors that can “taste” various substances such as sodium glutamate, glucose, carbohydrates, proteins, and fats and pass these tastes to the lateral hypothalamus and limbic system in the brain as a palatability signal through the vagus nerve.

The sense of taste may be distorted or disabled entirely by one or more types of taste disorders, including dysgeusia, hypogeusia, and ageusia. Dysgeusia, a distortion or alteration of taste, may be caused by chemotherapy and zinc deficiency. Hypogeusia, a partial loss of taste, may be caused by the chemotherapy drug bleomycin in some cases. Ageusia is the complete loss of one or more taste functions of the tongue, particularly the ability to detect sweetness, sourness, bitterness, saltiness, and umami. Ageusia may be caused by one or more of a variety of factors, including but not limited to: nerve tissue damage, especially damage to the lingual nerve and the glossopharyngeal nerve; neurological disorders such as Bell's palsy, familial dysautonomia, and multiple sclerosis; deficiency of vitamin B3 (niacin) and zinc; disorders of the endocrine system, such as Cushing's syndrome, hypothyroidism, and diabetes mellitus; and medicinal side-effects from antirheumatic drugs such as penicillamine, antiproliferative drugs such as cisplatin, ACE inhibitors, and other drugs including azelastine, clarithromycin and zopiclone.

The sensory-related proteins are typically selected based on an experimental association of the sensory-related protein to a sensory function including but not limited to nociception and taste, or to a sensory disorder such as a nociception disorder or a taste disorder described above. For example, the production rate or circulating concentration of a sensory-related protein may be elevated or depressed in a population having a sensory disorder, or the distribution of the sensory-related protein within the central or peripheral nervous system may be altered in a patient having a sensory disorder relative to a population or patient lacking the sensory disorder. Alternatively, the role of a protein in taste reception or nociception may be deduced by disrupting a biochemical pathway in which sensory protein is involved and observing the effects of this disruption on sensory function. Differences in protein levels may be assessed using proteomic techniques including but not limited to Western blot, immunohistochemical staining, enzyme linked immunosorbent assay (ELISA), and mass spectrometry. Alternatively, the sensory-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

Sensory-related proteins include but are not limited to nociception-related genes, pain-related genes, and taste-related genes. Non-limiting examples of nocioception-related genes include CALCA (calcitonin-related polypeptide alpha); FOS (FBJ murine osteosarcoma viral oncogene homolog); NPY (neuropeptide Y); TACR1 (tachykinin receptor 1); OPRM1 (opioid receptor mu 1); OPRD1 (opioid receptor delta 1); OPRK1 (opioid receptor kappa 1); TH (tyrosine hydroxylase); DRD2 (dopamine receptor D2); PTGS2 (prostaglandin-endoperoxide synthase 2 (prostaglandin G/H synthase and cyclooxygenase)); TNF (tumor necrosis factor (TNF superfamily member 2)); PDYN (prodynorphin); KNG1 (kininogen 1); CCK (cholecystokinin); NOS1 (nitric oxide synthase 1 (neuronal)); IL1B (interleukin 1 beta); SST (somatostatin); HTR3A (5-hydroxytryptamine (serotonin) receptor 3A); MAPK1 (mitogen-activated protein kinase 1); GAL (galanin prepropeptide); DYT10 (dystonia 10); TRPV1 (transient receptor potential cation channel subfamily V member 1); IL6 (interleukin 6 (interferon beta 2)); HTR2A (5-hydroxytryptamine (serotonin) receptor 2A); CNR1 (cannabinoid receptor 1 (brain)); NOS2 (nitric oxide synthase 2 inducible); PNOC (prepronociceptin); NTS (neurotensin); PTGS1 (prostaglandin-endoperoxide synthase 1 (prostaglandin G/H synthase and cyclooxygenase)); ACHE (acetylcholinesterase (Yt blood group)); NGF (nerve growth factor (beta polypeptide)); CCKBR (cholecystokinin B receptor); HTR1A (5-hydroxytryptamine (serotonin) receptor 1A); NPFF (neuropeptide FF-amide peptide precursor); CCL2 (chemokine (C-C motif) ligand 2); CAT (catalase); BDNF (brain-derived neurotrophic factor); ADORA1 (adenosine A1 receptor); NPR1 (natriuretic peptide receptor A/guanylate cyclase A (atrionatriuretic peptide receptor A)); GRP (gastrin-releasing peptide); MME (membrane metallo-endopeptidase); ABCB1 (ATP-binding cassette sub-family B (MDR/TAP) member 1); PENK (proenkephalin); TAC1 (tachykinin precursor 1); INS (insulin); NTRK1 (neurotrophic tyrosine kinase receptor type 1); SCN9A (sodium channel voltage-gated type IX alpha subunit); BCHE (butyrylcholinesterase); GALR2 (galanin receptor 2); ADCYAP1 (adenylate cyclase activating polypeptide 1 (pituitary)); HRH2 (histamine receptor H2); OXT (oxytocin prepropeptide); POMC (proopiomelanocortin); ADORA2A (adenosine A2a receptor); CPDX (coproporphyrinogen oxidase); NTSR2 (neurotensin receptor 2); SLC1A2 (solute carrier family 1 (glial high affinity glutamate transporter) member 2); OPRL1 (opiate receptor-like 1); GALR1 (galanin receptor 1); DDC (dopa decarboxylase (aromatic L-amino acid decarboxylase)); P2RX2 (purinergic receptor P2X ligand-gated ion channel 2); HMOX1 (heme oxygenase (decycling) 1); CNR2 (cannabinoid receptor 2 (macrophage)); HTR1 B (5-hydroxytryptamine (serotonin) receptor 1 B); HRH1 (histamine receptor H1); ADRA2A (adrenergic alpha-2A- receptor); GALR3 (galanin receptor 3); KCND1 (potassium voltage-gated channel Shal-related subfamily member 1); PRL (prolactin); IFNG (interferon gamma); GABBR1 (gamma-aminobutyric acid (GABA) B receptor 1); IL10 (interleukin 10); VWF (von Willebrand factor); GPT (glutamic-pyruvate transaminase (alanine am inotransferase)); CSF3 (colony stimulating factor 3 (granulocyte)); IL2 (interleukin 2); IFNA1 (interferon alpha 1); PROK1 (prokineticin 1); HMGCR (3-hydroxy-3-methylglutaryl-Coenzyme A reductase); JUN (jun oncogene); NPPA (natriuretic peptide precursor A); ADCY10 (adenylate cyclase 10 (soluble)); IL4 (interleukin 4); MAPK14 (mitogen-activated protein kinase 14); ADA (adenosine deaminase); TGFB1 (transforming growth factor beta 1); MAPK8 (mitogen-activated protein kinase 8); EDNRB (endothelin receptor type B); AKR1 B1 (aldo-keto reductase family 1 member B1 (aldose reductase)); NOS3 (nitric oxide synthase 3 (endothelial cell)); GABRE (gamma-aminobutyric acid (GABA) A receptor epsilon); KCNJ5 (potassium inwardly-rectifying channel subfamily J member 5); EPHX2 (epoxide hydrolase 2 cytoplasmic); EDNRA (endothelin receptor type A); NTSR1 (neurotensin receptor 1 (high affinity)); IL13 (interleukin 13); EDN3 (endothelin 3); CRH (corticotropin releasing hormone); PPARA (peroxisome proliferator-activated receptor alpha); CCKAR (cholecystokinin A receptor); FAAH (fatty acid amide hydrolase); EDN1 (endothelin 1); CABIN1 (calcineurin binding protein 1); NTRK3 (neurotrophic tyrosine kinase receptor type 3); NTF3 (neurotrophin 3); PL-5283 (PL-5283 protein); APC (adenomatous polyposis coli); DBH (dopamine beta-hydroxylase (dopamine beta-monooxygenase)); SYP (synaptophysin); SLC8A1 (solute carrier family 8 (sodium/calcium exchanger) member 1); CHRNA4 (cholinergic receptor nicotinic alpha 4); TRPA1 (transient receptor potential cation channel subfamily A member 1); CYBB (cytochrome b-245 beta polypeptide); RAC1 (ras-related C3 botulinum toxin substrate 1 (rho family small GTP binding protein Rac1)); IDS (iduronate 2-sulfatase); LTF (lactotransferrin); TRPM8 (transient receptor potential cation channel subfamily M member 8); MRGPRX3 (MAS-related GPR member X3); CCR5 (chemokine (C-C motif) receptor 5); CCL5 (chemokine (C-C motif) ligand 5); MBL2 (mannose-binding lectin (protein C) 2 soluble (opsonic defect)); P2RX3 (purinergic receptor P2X ligand-gated ion channel 3); MRGPRX2 (MAS-related GPR member X2); FAM134B (family with sequence similarity 134 member B); IL8 (interleukin 8); NTRK2 (neurotrophic tyrosine kinase receptor type 2); GJA1 (gap junction protein alpha 1 43kDa); CACNA1 H (calcium channel voltage-dependent T type alpha 1H subunit); HDC (histidine decarboxylase); IFT88 (intraflagellar transport 88 homolog (Chlamydomonas)); POU4F3 (POU class 4 homeobox 3); ATOH1 (atonal homolog 1 (Drosophila)); GRM3 (glutamate receptor metabotropic 3); ADK (adenosine kinase); RIPK2 (receptor-interacting serine-threonine kinase 2); ANPEP (alanyl (membrane) aminopeptidase); DRD1 (dopamine receptor D1); NFE2L2 (nuclear factor (erythroid-derived 2)-like 2); RET (ret proto-oncogene); AHSP (alpha hemoglobin stabilizing protein); ESR2 (estrogen receptor 2 (ER beta)); HLA-A (major histocompatibility complex class I A); CHRM2 (cholinergic receptor muscarinic 2); ALAD (aminolevulinate delta-dehydratase); CXCL2 (chemokine (C-X-C motif) ligand 2); HSPG2 (heparan sulfate proteoglycan 2); F2R (coagulation factor II (thrombin) receptor); KCNIP3 (Kv channel interacting protein 3 calsenilin); GRIN1 (glutamate receptor ionotropic N-methyl D-aspartate 1); GRIK1 (glutamate receptor ionotropic kainate 1); P2RX7 (purinergic receptor P2X ligand-gated ion channel 7); CACNA1 B (calcium channel voltage-dependent N type alpha 1B subunit); TACR2 (tachykinin receptor 2); NPFFR2 (neuropeptide FF receptor 2); MRGPRX1 (MAS-related GPR member X1); MRGPRX4 (MAS-related GPR member X4); PTH2 (parathyroid hormone 2); DRGX (dorsal root ganglia homeobox); CCR3 (chemokine (C-C motif) receptor 3); CYBA (cytochrome b-245 alpha polypeptide); CCL7 (chemokine (C-C motif) ligand 7); S100A6 (S100 calcium binding protein A6); CHGA (chromogranin A (parathyroid secretory protein 1)); CCL4 (chemokine (C-C motif) ligand 4); HTR5A (5-hydroxytryptamine (serotonin) receptor 5A); KCNC3 (potassium voltage-gated channel Shaw-related subfamily member 3); PNMT (phenylethanolamine N-methyltransferase); CCL8 (chemokine (C-C motif) ligand 8); LTB4R (leukotriene B4 receptor); NOXA1 (NADPH oxidase activator 1); PHOX2B (paired-like homeobox 2b); NOX1 (NADPH oxidase 1); NOX4 (NADPH oxidase 4); TAS1 R3 (taste receptor type 1 member 3); NEUROG1 (neurogenin 1); NOXO1 (NADPH oxidase organizer 1); TRIM26 (tripartite motif-containing 26); OMP (olfactory marker protein); ZC3H12A (zinc finger CCCH-type containing 12A); CXCR4 (chemokine (C-X-C motif) receptor 4); PLA2G2A (phospholipase A2 group IIA (platelets synovial fluid)); PLA2G1 B (phospholipase A2 group IB (pancreas)); GNRH1 (gonadotropin-releasing hormone 1 (luteinizing-releasing hormone)); TJP1 (tight junction protein 1 (zona occludens 1)); NRG1 (neuregulin 1); GRIN2B (glutamate receptor ionotropic N-methyl D-aspartate 2B); COL18A1 (collagen type XVIII alpha 1); HTR6 (5-hydroxytryptamine (serotonin) receptor 6); HTR7 (5-hydroxytryptamine (serotonin) receptor 7 (adenylate cyclase-coupled)); SLC1A3 (solute carrier family 1 (glial high affinity glutamate transporter) member 3); CACNA1 D (calcium channel voltage-dependent L type alpha 1D subunit); GRM2 (glutamate receptor metabotropic 2); HNMT (histamine N-methyltransferase); ADORA2B (adenosine A2b receptor); SLC1A1 (solute carrier family 1 (neuronal/epithelial high affinity glutamate transporter system Xag) member 1); GABBR2 (gamma-aminobutyric acid (GABA) B receptor 2); PCSK2 (proprotein convertase subtilisin/kexin type 2); CD160 (CD160 molecule); TSPO (translocator protein (18 kDa)); NPSR1 (neuropeptide S receptor 1); PROL1 (proline rich lacrimal 1); NPVF (neuropeptide VF precursor); NPS (neuropeptide S); PRNP (prion protein); GRIA2 (glutamate receptor ionotropic AMPA 2); GRIA1 (glutamate receptor ionotropic AMPA 1); PRKCE (protein kinase C epsilon); ITPR1 (inositol 1 (4 (5-triphosphate receptor type 1); CBR1 (carbonyl reductase 1); ADORA3 (adenosine A3 receptor); FMR1 (fragile X mental retardation 1); ALOX5 (arachidonate 5-lipoxygenase); GRM7 (glutamate receptor metabotropic 7); PRKG1 (protein kinase cGMP-dependent type I); IL7 (interleukin 7); GRIK5 (glutamate receptor ionotropic kainate 5); HCRTR1 (hypocretin (orexin) receptor 1); CCL21 (chemokine (C-C motif) ligand 21); URN (interleukin 1 receptor antagonist); CX3CR1 (chemokine (C-X3-C motif) receptor 1); P2RX4 (purinergic receptor P2X ligand-gated ion channel 4); AVP (arginine vasopressin); PRPH (peripherin); MTOR (mechanistic target of rapamycin (serine/threonine kinase)); NFATC4 (nuclear factor of activated T-cells cytoplasmic calcineurin-dependent 4); F2RL1 (coagulation factor II (thrombin) receptor-like 1); EDN2 (endothelin 2); ACCN2 (amiloride-sensitive cation channel 2 neuronal); P2RX1 (purinergic receptor P2X ligand-gated ion channel 1); ENPEP (glutamyl aminopeptidase (aminopeptidase A)); CLDN5 (claudin 5); GFRA3 (GDNF family receptor alpha 3); PTGER1 (prostaglandin E receptor 1 (subtype EP1) 42kDa); OCLN (occludin); P2RX5 (purinergic receptor P2X ligand-gated ion channel 5); CALB1 (calbindin 1 28kDa); CXCL1 (chemokine (C-X-C motif) ligand 1 (melanoma growth stimulating activity alpha)); BDKRB1 (bradykinin receptor B1); TRPV4 (transient receptor potential cation channel subfamily V member 4); PRLHR (prolactin releasing hormone receptor); P2RX6 (purinergic receptor P2X ligand-gated ion channel 6); LALBA (lactalbumin alpha-); IL17A (interleukin 17A); NPFFR1 (neuropeptide FF receptor 1); ARTN (artemin); PTH2R (parathyroid hormone 2 receptor); PROK2 (prokineticin 2); PROKR2 (prokineticin receptor 2); MAS1 L (MAS1 oncogene-like); PROKR1 (prokineticin receptor 1); MRGPRD (MAS-related GPR member D); MRGPRE (MAS-related GPR member E); MRGPRF (MAS-related GPR member F); and PRLH (prolactin releasing hormone).

Non-limiting examples of pain-related genes include PTGS2 (prostaglandin-endoperoxide synthase 2 (prostaglandin G/H synthase and cyclooxygenase)); SCN9A (sodium channel voltage-gated type IX alpha subunit); TRPV1 (transient receptor potential cation channel subfamily V member 1); KNG1 (kininogen 1); IL1 B (interleukin 1 beta); NTRK1 (neurotrophic tyrosine kinase receptor type 1); BDKRB1 (bradykinin receptor B1); BDKRB2 (bradykinin receptor B2); P2RX3 (purinergic receptor P2X ligand-gated ion channel 3); POMC (proopiomelanocortin); GAL (galanin prepropeptide); SCN10A (sodium channel voltage-gated type X alpha subunit); PRKCG (protein kinase C gamma); PTGS1 (prostaglandin-endoperoxide synthase 1 (prostaglandin G/H synthase and cyclooxygenase)); GRIN1 (glutamate receptor ionotropic N-methyl D-aspartate 1); NGF (nerve growth factor (beta polypeptide)); CALCA (calcitonin-related polypeptide alpha); TNF (tumor necrosis factor (TNF superfamily member 2)); IL6 (interleukin 6 (interferon beta 2)); CRP (C-reactive protein pentraxin-related); INS (insulin); OPRM1 (opioid receptor mu 1); COMT (catechol-O-methyltransferase); CNR1 (cannabinoid receptor 1 (brain)); IL10 (interleukin 10); CCK (cholecystokinin); TACR1 (tachykinin receptor 1); OPRD1 (opioid receptor delta 1); NPFFR2 (neuropeptide FF receptor 2); TGFB1 (transforming growth factor beta 1); NOS1 (nitric oxide synthase 1 (neuronal)); CRH (corticotropin releasing hormone); GALR3 (galanin receptor 3); MSD (microcephaly with spastic diplegia (Paine syndrome)); IL8 (interleukin 8); MB (myoglobin); DYT10 (dystonia 10); PRL (prolactin); MAPK1 (mitogen-activated protein kinase 1); TAC1 (tachykinin precursor 1); PDYN (prodynorphin); GCH1 (GTP cyclohydrolase 1); SOD1 (superoxide dismutase 1 soluble); SLC6A4 (solute carrier family 6 (neurotransmitter transporter serotonin) member 4); GRIN2B (glutamate receptor ionotropic N-methyl D-aspartate 2B); NPY (neuropeptide Y); OPRK1 (opioid receptor kappa 1); PENK (proenkephalin); TRPA1 (transient receptor potential cation channel subfamily A member 1); IL2 (interleukin 2); CABIN1 (calcineurin binding protein 1); NOS2 (nitric oxide synthase 2 inducible); PNOC (prepronociceptin); GRIN2A (glutamate receptor ionotropic N-methyl D-aspartate 2A); CHKA (choline kinase alpha); FOS (FBJ murine osteosarcoma viral oncogene homolog); GRIN2D (glutamate receptor ionotropic N-methyl D-aspartate 2D); CCL2 (chemokine (C-C motif) ligand 2); HTR2A (5-hydroxytryptamine (serotonin) receptor 2A); CYP19A1 (cytochrome P450 family 19 subfamily A polypeptide 1); GRIN2C (glutamate receptor ionotropic N-methyl D-aspartate 2C); PTGES (prostaglandin E synthase); HTR3A (5-hydroxytryptamine (serotonin) receptor 3A); FAAH (fatty acid amide hydrolase); NTRK2 (neurotrophic tyrosine kinase receptor type 2); ACE (angiotensin I converting enzyme (peptidyl-dipeptidase A) 1); GRM1 (glutamate receptor metabotropic 1); GDNF (glial cell derived neurotrophic factor); TLR4 (toll-like receptor 4); DRD2 (dopamine receptor D2); GRM5 (glutamate receptor metabotropic 5); VIP (vasoactive intestinal peptide); PROK1 (prokineticin 1); GALR2 (galanin receptor 2); ESR1 (estrogen receptor 1); NR3C1 (nuclear receptor subfamily 3 group C member 1 (glucocorticoid receptor)); MME (membrane metallo-endopeptidase); EDN1 (endothelin 1); NPY1 R (neuropeptide Y receptor Y1); ADK (adenosine kinase); NPY2R (neuropeptide Y receptor Y2); GALR1 (galanin receptor 1); TRPC1 (transient receptor potential cation channel subfamily C member 1); TRPC5 (transient receptor potential cation channel subfamily C member 5); TRPC6 (transient receptor potential cation channel subfamily C member 6); HBS1 L (HBS1-like (S. cerevisiae)); GRIN3A (glutamate receptor ionotropic N-methyl-D-aspartate 3A); GRIN3B (glutamate receptor ionotropic N-methyl-D-aspartate 3B); GPR55 (G protein-coupled receptor 55); MRGPRX3 (MAS-related GPR member X3); HSN2 (hereditary sensory neuropathy type II); AKR1 B1 (aldo-keto reductase family 1 member B1 (aldose reductase)); NGFR (nerve growth factor receptor (TNFR superfamily member 16)); PRKCE (protein kinase C epsilon); TRPM8 (transient receptor potential cation channel subfamily M member 8); SST (somatostatin); URN (interleukin 1 receptor antagonist); CD4OLG (CD40 ligand); BCHE (butyrylcholinesterase); ACPP (acid phosphatase prostate); NPPC (natriuretic peptide precursor C); SCN11A (sodium channel voltage-gated type XI alpha subunit); KLK3 (kallikrein-related peptidase 3); PTGIR (prostaglandin I2 (prostacyclin) receptor (IP)); PPYR1 (pancreatic polypeptide receptor 1); NPY5R (neuropeptide Y receptor Y5); NPFFR1 (neuropeptide FF receptor 1); ACCN4 (amiloride-sensitive cation channel 4 pituitary); MMEL1 (membrane metallo-endopeptidase-like 1); UCN (urocortin); IFNG (interferon gamma); CYP2D6 (cytochrome P450 family 2 subfamily D polypeptide 6); CACNA1B (calcium channel voltage-dependent N type alpha 1B subunit); ACCN3 (amiloride-sensitive cation channel 3); BDNF (brain-derived neurotrophic factor); MAPK14 (mitogen-activated protein kinase 14); CNR2 (cannabinoid receptor 2 (macrophage)); MMP9 (matrix metallopeptidase 9 (gelatinase B 92kDa gelatinase 92kDa type IV collagenase)); IL4 (interleukin 4); ADRB2 (adrenergic beta-2- receptor surface); GFAP (glial fibrillary acidic protein); KCNIP3 (Kv channel interacting protein 3 calsenilin); IL1R1 (interleukin 1 receptor type I); ABCB1 (ATP-binding cassette sub-family B (MDR/TAP) member 1); MAPK8 (mitogen-activated protein kinase 8); MC1R (melanocortin 1 receptor (alpha melanocyte stimulating hormone receptor)); ALB (albumin); CAMK2G (calcium/calmodulin-dependent protein kinase II gamma); PLAT (plasminogen activator tissue); P2RX4 (purinergic receptor P2X ligand-gated ion channel 4); MAPK3 (mitogen-activated protein kinase 3); TNFRSF1A (tumor necrosis factor receptor superfamily member 1A); TTF2 (transcription termination factor RNA polymerase II); ITIH4 (inter-alpha (globulin) inhibitor H4 (plasma Kallikrein-sensitive glycoprotein)); CXCR4 (chemokine (C-X-C motif) receptor 4); SOD2 (superoxide dismutase 2 mitochondrial); SRC (v-src sarcoma (Schmidt-Ruppin A-2) viral oncogene homolog (avian)); PPARA (peroxisome proliferator-activated receptor alpha); CREB1 (cAMP responsive element binding protein 1); F2 (coagulation factor II (thrombin)); GAD1 (glutamate decarboxylase 1 (brain 67kDa)); P2RX7 (purinergic receptor P2X ligand-gated ion channel 7); F3 (coagulation factor III (thromboplastin tissue factor)); MIF (macrophage migration inhibitory factor (glycosylation-inhibiting factor)); LEP (leptin); GNRH1 (gonadotropin-releasing hormone 1 (luteinizing-releasing hormone)); OPRL1 (opiate receptor-like 1); CCL3 (chemokine (C-C motif) ligand 3); UCP1 (uncoupling protein 1 (mitochondrial proton carrier)); NTS (neurotensin); SLC12A5 (solute carrier family 12 (potassium/chloride transporter) member 5); CD160 (CD160 molecule); NPFF (neuropeptide FF-amide peptide precursor); ANPEP (alanyl (membrane) aminopeptidase); VDR (vitamin D (1 (25- dihydroxyvitamin D3) receptor); JUN (jun oncogene); ADIPOQ (adiponectin C1Q and collagen domain containing); ELK1 (ELK1 member of ETS oncogene family); FGF2 (fibroblast growth factor 2 (basic)); GABBR1 (gamma-aminobutyric acid (GABA) B receptor 1); COMP (cartilage oligomeric matrix protein); SERPINE1 (serpin peptidase inhibitor clade E (nexin plasminogen activator inhibitor type 1) member 1); GRM2 (glutamate receptor metabotropic 2); GAD2 (glutamate decarboxylase 2 (pancreatic islets and brain 65kDa)); EPO (erythropoietin); NTF3 (neurotrophin 3); IL1 R2 (interleukin 1 receptor type II); ADCY1 (adenylate cyclase 1 (brain)); PEPD (peptidase D); HBEGF (heparin-binding EGF-like growth factor); GAST (gastrin); KCND1 (potassium voltage-gated channel Shal-related subfamily member 1); OXT (oxytocin prepropeptide); SLC17A5 (solute carrier family 17 (anion/sugar transporter) member 5); PL-5283 (PL-5283 protein); STN (statin); EGF (epidermal growth factor (beta-urogastrone)); CACNA1A (calcium channel voltage-dependent P/Q type alpha 1A subunit); VWF (von Willebrand factor); ANXA5 (annexin A5); MMP2 (matrix metallopeptidase 2 (gelatinase A 72kDa gelatinase 72kDa type IV collagenase)); HMGCR (3-hydroxy-3-methylglutaryl-Coenzyme A reductase); SPP1 (secreted phosphoprotein 1); SCNSA (sodium channel voltage-gated type V alpha subunit); GLA (galactosidase alpha); CHRNA4 (cholinergic receptor nicotinic alpha 4); PITX2 (paired-like homeodomain 2); DLG4 (discs large homolog 4 (Drosophila)); GNB3 (guanine nucleotide binding protein (G protein) beta polypeptide 3); ADORA1 (adenosine Al receptor); MYH7 (myosin heavy chain 7 cardiac muscle beta); TXN (thioredoxin); CP (ceruloplasmin (ferroxidase)); CSF3 (colony stimulating factor 3 (granulocyte)); SLC1A1 (solute carrier family 1 (neuronal/epithelial high affinity glutamate transporter system Xag) member 1); IAPP (islet amyloid polypeptide); GUK1 (guanylate kinase 1); NPPA (natriuretic peptide precursor A); ADCYAP1 (adenylate cyclase activating polypeptide 1 (pituitary)); XDH (xanthine dehydrogenase); SRD5A1 (steroid-5-alpha-reductase alpha polypeptide 1 (3-oxo-5 alpha-steroid delta 4-dehydrogenase alpha 1)); IDO1 (indoleamine 2 (3-dioxygenase 1); REN (renin); CX3CL1 (chemokine (C-X3-C motif) ligand 1); NEK3 (NIMA (never in mitosis gene a)-related kinase 3); KIAA0101 (KIAA0101); ARTN (artemin); SLC17A6 (solute carrier family 17 (sodium-dependent inorganic phosphate cotransporter) member 6); GPR172B (G protein-coupled receptor 172B); BCL2 (B-cell CLL/lymphoma 2); CREBBP (CREB binding protein); NCAM1 (neural cell adhesion molecule 1); EPOR (erythropoietin receptor); ATP2A2 (ATPase Ca++transporting cardiac muscle slow twitch 2); HTR7 (5-hydroxytryptamine (serotonin) receptor 7 (adenylate cyclase-coupled)); MYH11 (myosin heavy chain 11 smooth muscle); AGTR2 (angiotensin II receptor type 2); ENO2 (enolase 2 (gamma neuronal)); VIM (vimentin); MAP2K3 (mitogen-activated protein kinase kinase 3); ADAM17 (ADAM metallopeptidase domain 17); IL6ST (interleukin 6 signal transducer (gp130 oncostatin M receptor)); PSMA2 (proteasome (prosome macropain) subunit alpha type 2); MAP2K6 (mitogen-activated protein kinase kinase 6); S100A9 (S100 calcium binding protein A9); S100A8 (S100 calcium binding protein A8); CCL21 (chemokine (C-C motif) ligand 21); EPHA4 (EPH receptor A4); ADCYAP1 R1 (adenylate cyclase activating polypeptide 1 (pituitary) receptor type I); CGB (chorionic gonadotropin beta polypeptide); IBSP (integrin-binding sialoprotein); SORT1 (sortilin 1); CNTF (ciliary neurotrophic factor); DAO (D-amino-acid oxidase); NRTN (neurturin); HCRT (hypocretin (orexin) neuropeptide precursor); MAP1 B (microtubule-associated protein 1 B); ADAMTS13 (ADAM metallopeptidase with thrombospondin type 1 motif 13); ABP1 (amiloride binding protein 1 (amine oxidase (coppercontaining))); SLC17A7 (solute carrier family 17 (sodium-dependent inorganic phosphate cotransporter) member 7); CADM1 (cell adhesion molecule 1); AIF1 (allograft inflammatory factor 1); ADCY10 (adenylate cyclase 10 (soluble)); TRIM26 (tripartite motif-containing 26); GGT2 (gamma-glutamyltransferase 2); ILIA (interleukin 1 alpha); C1S (complement component 1 s subcomponent); MPO (myeloperoxidase); NPPB (natriuretic peptide precursor B); F2RL1 (coagulation factor II (thrombin) receptor-like 1); TNNI3 (troponin I type 3 (cardiac)); SELP (selectin P (granule membrane protein 140kDa antigen CD62)); TNFRSF11B (tumor necrosis factor receptor superfamily member 11 b); FABP3 (fatty acid binding protein 3 muscle and heart (mammary-derived growth inhibitor)); ADRA2A (adrenergic alpha-2A- receptor); HTR1A (5-hydroxytryptamine (serotonin) receptor 1A); CASP3 (caspase 3 apoptosis-related cysteine peptidase); CPDX (coproporphyrinogen oxidase); SCN7A (sodium channel voltage-gated type VII alpha); PPARG (peroxisome proliferator-activated receptor gamma); MYL3 (myosin light chain 3 alkali; ventricular skeletal slow); CRHR1 (corticotropin releasing hormone receptor 1); ICAM1 (intercellular adhesion molecule 1); MAPK10 (mitogen-activated protein kinase 10); CAMK2A (calcium/calmodulin-dependent protein kinase II alpha); EDNRB (endothelin receptor type B); CSF2 (colony stimulating factor 2 (granulocyte-macrophage)); SCN4A (sodium channel voltage-gated type IV alpha subunit); EPRS (glutamyl-prolyl-tRNA synthetase); HBB (hemoglobin beta); IL5 (interleukin 5 (colony-stimulating factor eosinophil)); EDNRA (endothelin receptor type A); MEFV (Mediterranean fever); PAPPA (pregnancy-associated plasma protein A pappalysin 1); PTGER4 (prostaglandin E receptor 4 (subtype EP4)); PIK3C2A (phosphoinositide-3-kinase class 2 alpha polypeptide); BGLAP (bone gamma-carboxyglutamate (gla) protein); POR (P450 (cytochrome) oxidoreductase); NOS3 (nitric oxide synthase 3 (endothelial cell)); PRKACA (protein kinase cAMP-dependent catalytic alpha); TP53 (tumor protein p53); RPS6KB1 (ribosomal protein S6 kinase 70kDa polypeptide 1); PRKAR1A (protein kinase cAMP-dependent regulatory type I alpha (tissue specific extinguisher 1)); IGF1 (insulin-like growth factor 1 (somatomedin C)); GRIA2 (glutamate receptor ionotropic AMPA 2); GRIA1 (glutamate receptor ionotropic AMPA 1); IL13 (interleukin 13); HSP9OAA1 (heat shock protein 90kDa alpha (cytosolic) class A member 1); PIK3CG (phosphoinositide-3-kinase catalytic gamma polypeptide); IL12B (interleukin 12B (natural killer cell stimulatory factor 2 cytotoxic lymphocyte maturation factor 2 p40)); CYP3A4 (cytochrome P450 family 3 subfamily A polypeptide 4); PRKACB (protein kinase cAMP-dependent catalytic beta); PRKAR2A (protein kinase cAMP-dependent regulatory type II alpha); GRM8 (glutamate receptor metabotropic 8); CAMK2D (calcium/calmodulin-dependent protein kinase II delta); GRM7 (glutamate receptor metabotropic 7); GH1 (growth hormone 1); TNNT2 (troponin T type 2 (cardiac)); MAOA (monoamine oxidase A); CAMK2B (calcium/calmodulin-dependent protein kinase II beta); SERPINC1 (serpin peptidase inhibitor clade C (antithrombin) member 1); SLC12A2 (solute carrier family 12 (sodium/potassium/chloride transporters) member 2); COL2A1 (collagen type II alpha 1); PRKAR1B (protein kinase cAMP-dependent regulatory type I beta); CX3CR1 (chemokine (C-X3-C motif) receptor 1); PRKACG (protein kinase cAMP-dependent catalytic gamma); SLC6A2 (solute carrier family 6 (neurotransmitter transporter noradrenalin) member 2); MTOR (mechanistic target of rapamycin (serine/threonine kinase)); DLG2 (discs large homolog 2 (Drosophila)); MGLL (monoglyceride lipase); ATF3 (activating transcription factor 3); ALPP (alkaline phosphatase placental (Regan isozyme)); COL9A2 (collagen type IX alpha 2); HBG2 (hemoglobin gamma G); MRGPRX1 (MAS-related GPR member X1); FGFR1 (fibroblast growth factor receptor 1); NFKB1 (nuclear factor of kappa light polypeptide gene enhancer in B-cells 1); EIF4E (eukaryotic translation initiation factor 4E); PRKCA (protein kinase C alpha); EGFR (epidermal growth factor receptor (erythroblastic leukemia viral (v-erb-b) oncogene homolog avian)); PIK3R1 (phosphoinositide-3-kinase regulatory subunit 1 (alpha)); PTPN6 (protein tyrosine phosphatase non-receptor type 6); PLCG2 (phospholipase C gamma 2 (phosphatidylinositol-specific)); PRKCQ (protein kinase C theta); PLG (plasminogen); GRIA3 (glutamate receptor ionotrophic AMPA 3); IL6R (interleukin 6 receptor); HIF1A (hypoxia inducible factor 1 alpha subunit (basic helix-loop-helix transcription factor)); ALPL (alkaline phosphatase liver/bone/kidney); ADCY6 (adenylate cyclase 6); PRKCZ (protein kinase C zeta); GRM3 (glutamate receptor metabotropic 3); IL2RA (interleukin 2 receptor alpha); PIK3CD (phosphoinositide-3-kinase catalytic delta polypeptide); SNCA (synuclein alpha (non A4 component of amyloid precursor)); CYP1A1 (cytochrome P450 family 1 subfamily A polypeptide 1); PLCG1 (phospholipase C gamma 1); DBH (dopamine beta-hydroxylase (dopamine beta-monooxygenase)); GRIK1 (glutamate receptor ionotropic kainate 1); PRKCH (protein kinase C eta); PRKCD (protein kinase C delta); CAT (catalase); ITPR1 (inositol 1 (4 (5-triphosphate receptor type 1); PLCB3 (phospholipase C beta 3 (phosphatidylinositol-specific)); PLCB2 (phospholipase C beta 2); PIK3CB (phosphoinositide-3-kinase catalytic beta polypeptide); PLA2G2A (phospholipase A2 group IIA (platelets synovial fluid)); PIK3CA (phosphoinositide-3-kinase catalytic alpha polypeptide); DRD3 (dopamine receptor D3); DMD (dystrophin); MAPK7 (mitogen-activated protein kinase 7); PIK3C3 (phosphoinositide-3-kinase class 3); LPL (lipoprotein lipase); ADCY8 (adenylate cyclase 8 (brain)); HSPG2 (heparan sulfate proteoglycan 2); CCL5 (chemokine (C-C motif) ligand 5); ALOX5 (arachidonate 5-lipoxygenase); PRKCI (protein kinase C iota); PRKAR2B (protein kinase cAMP-dependent regulatory type II beta); GLRA1 (glycine receptor alpha 1); MMP12 (matrix metallopeptidase 12 (macrophage elastase)); CHAT (choline acetyltransferase); LRP5 (low density lipoprotein receptor-related protein 5); TIMP1 (TIMP metallopeptidase inhibitor 1); PLCB1 (phospholipase C beta 1 (phosphoinositide-specific)); F2R (coagulation factor II (thrombin) receptor); EIF2S1 (eukaryotic translation initiation factor 2 subunit 1 alpha 35kDa); SELL (selectin L); THBS2 (thrombospondin 2); ADRA2C (adrenergic alpha-2C- receptor); HTR2B (5-hydroxytryptamine (serotonin) receptor 2B); TF (transferrin); CST3 (cystatin C); PIK3C2B (phosphoinositide-3-kinase class 2 beta polypeptide); PLCD1 (phospholipase C delta 1); PLCB4 (phospholipase C beta 4); NR1 I2 (nuclear receptor subfamily 1 group I member 2); PIK3R2 (phosphoinositide-3-kinase regulatory subunit 2 (beta)); PYGM (phosphorylase glycogen muscle); KCNQ3 (potassium voltage-gated channel KQT-like subfamily member 3); PECAM1 (platelet/endothelial cell adhesion molecule); CCL4 (chemokine (C-C motif) ligand 4); TACR3 (tachykinin receptor 3); GRM4 (glutamate receptor metabotropic 4); 9-Sep (septin 9); LBP (lipopolysaccharide binding protein); CAMK1 (calcium/calmodulin-dependent protein kinase I); SCN1A (sodium channel voltage-gated type I alpha subunit); OSM (oncostatin M); SQSTM1 (sequestosome 1); AVP (arginine vasopressin); PRPH (peripherin); GLRA3 (glycine receptor alpha 3); PIK3R3 (phosphoinositide-3-kinase regulatory subunit 3 (gamma)); PTGER3 (prostaglandin E receptor 3 (subtype EP3)); SPTLC1 (serine palmitoyltransferase long chain base subunit 1); PIK3C2G (phosphoinositide-3-kinase class 2 gamma polypeptide); PTH (parathyroid hormone); TJP1 (tight junction protein 1 (zona occludens 1)); SCN2B (sodium channel voltage-gated type II beta); EIF2AK2 (eukaryotic translation initiation factor 2-alpha kinase 2); CACNA2D2 (calcium channel voltage-dependent alpha 2/delta subunit 2); ADCY5 (adenylate cyclase 5); PRKCB (protein kinase C beta); TAT (tyrosine aminotransferase); CLDN5 (claudin 5); HYAL1 (hyaluronoglucosaminidase 1); PLCD3 (phospholipase C delta 3); PTGER1 (prostaglandin E receptor 1 (subtype EP1) 42 kDa); KRT7 (keratin 7); PPIG (peptidylprolyl isomerase G (cyclophilin G)); OCLN (occludin); CACNA2D1 (calcium channel voltage-dependent alpha 2/delta subunit 1); CXCL1 (chemokine (C-X-C motif) ligand 1 (melanoma growth stimulating activity alpha)); SLC6A1 (solute carrier family 6 (neurotransmitter transporter GABA) member 1); SERPINA6 (serpin peptidase inhibitor clade A (alpha-1 antiproteinase antitrypsin) member 6); TRPV4 (transient receptor potential cation channel subfamily V member 4); NNT (nicotinamide nucleotide transhydrogenase); GRM6 (glutamate receptor metabotropic 6); DPP3 (dipeptidyl-peptidase 3); SLC18A3 (solute carrier family 18 (vesicular acetylcholine) member 3); GPT (glutamic-pyruvate transaminase (alanine aminotransferase)); TFIP11 (tuftelin interacting protein 11); KCNK2 (potassium channel subfamily K member 2); CYB5A (cytochrome b5 type A (microsomal)); PLCZ1 (phospholipase C zeta 1); ANK3 (ankyrin 3 node of Ranvier (ankyrin G)); BLVRB (biliverdin reductase B (flavin reductase (NADPH))); FGF23 (fibroblast growth factor 23); CAMK1G (calcium/calmodulin-dependent protein kinase IG); TRPV2 (transient receptor potential cation channel subfamily V member 2); PIK3R5 (phosphoinositide-3-kinase regulatory subunit 5); GRINA (glutamate receptor ionotropic N-methyl D-aspartate-associated protein 1 (glutamate binding)); PROK2 (prokineticin 2); ENAM (enamelin); NPBWR1 (neuropeptides B/W receptor 1); LXN (latexin); MRGPRX2 (MAS-related GPR member X2); AMBN (ameloblastin (enamel matrix protein)); UCN2 (urocortin 2); TUFT1 (tuftelin 1); FAM134B (family with sequence similarity 134 member B); TAC4 (tachykinin 4 (hemokinin)); NPB (neuropeptide B); PDGFRB (platelet-derived growth factor receptor beta polypeptide); ITGB2 (integrin beta 2 (complement component 3 receptor 3 and 4 subunit)); FGFR2 (fibroblast growth factor receptor 2); TSC1 (tuberous sclerosis 1); RUNX1 (runt-related transcription factor 1); PTPRC (protein tyrosine phosphatase receptor type C); FYN (FYN oncogene related to SRC FGR YES); APP (amyloid beta (A4) precursor protein); PGR (progesterone receptor); ERBB2 (v-erb-b2 erythroblastic leukemia viral oncogene homolog 2 neuro/glioblastoma derived oncogene homolog (avian)); ERBB3 (v-erb-b2 erythroblastic leukemia viral oncogene homolog 3 (avian)); CSTB (cystatin B (stefin B)); CASP8 (caspase 8 apoptosis-related cysteine peptidase); ADA (adenosine deaminase); WT1 (Wilms tumor 1); CD44 (CD44 molecule (Indian blood group)); NFKBIA (nuclear factor of kappa light polypeptide gene enhancer in B-cells inhibitor alpha); RB1 (retinoblastoma 1); S100B (S100 calcium binding protein B); MYL2 (myosin light chain 2 regulatory cardiac slow); PSEN1 (presenilin 1); EGR1 (early growth response 1); GJA1 (gap junction protein alpha 1 43kDa); SLC6A3 (solute carrier family 6 (neurotransmitter transporter dopamine) member 3); JAK2 (Janus kinase 2); RYR1 (ryanodine receptor 1 (skeletal)); CCKBR (cholecystokinin B receptor); RELA (v-rel reticuloendotheliosis viral oncogene homolog A (avian)); RET (ret proto-oncogene); ANXA2 (annexin A2); CCR5 (chemokine (C-C motif) receptor 5); TGFBR1 (transforming growth factor beta receptor 1); PARK2 (Parkinson disease (autosomal recessive juvenile) 2 parkin); ITGA6 (integrin alpha 6); DPYD (dihydropyrimidine dehydrogenase); TH (tyrosine hydroxylase); GNAS (GNAS complex locus); TNFRSF1B (tumor necrosis factor receptor superfamily member 1B); COL1A1 (collagen type I alpha 1); HMOX1 (heme oxygenase (decycling) 1); LDHA (lactate dehydrogenase A); MBP (myelin basic protein); SERPINA1 (serpin peptidase inhibitor clade A (alpha-1 antiproteinase antitrypsin) member 1); SCNN1A (sodium channel nonvoltage-gated 1 alpha); ACTN2 (actinin alpha 2); ACHE (acetylcholinesterase (Yt blood group)); TTN (titin); CCNH (cyclin H); SLC1A2 (solute carrier family 1 (glial high affinity glutamate transporter) member 2); ESR2 (estrogen receptor 2 (ER beta)); HTR4 (5-hydroxytryptamine (serotonin) receptor 4); KCNH2 (potassium voltage-gated channel subfamily H (eag-related) member 2); ADRBK1 (adrenergic beta receptor kinase 1); IRS1 (insulin receptor substrate 1); C3 (complement component 3); LTA4H (leukotriene A4 hydrolase); GSR (glutathione reductase); NF2 (neurofibromin 2 (merlin)); ATF2 (activating transcription factor 2); IGFBP3 (insulin-like growth factor binding protein 3); BMP4 (bone morphogenetic protein 4); CDK5 (cyclin-dependent kinase 5); CDC25C (cell division cycle 25 homolog C (S. pombe)); CD36 (CD36 molecule (thrombospondin receptor)); TPM1 (tropomyosin 1 (alpha)); CD40 (CD40 molecule TNF receptor superfamily member 5); CYP1A2 (cytochrome P450 family 1 subfamily A polypeptide 2); FN1 (fibronectin 1); PKM2 (pyruvate kinase muscle); G6PD (glucose-6-phosphate dehydrogenase); CGA (glycoprotein hormones alpha polypeptide); HSF1 (heat shock transcription factor 1); CD3E (CD3e molecule epsilon (CD3-TCR complex)); CYP3A5 (cytochrome P450 family 3 subfamily A polypeptide 5); CYP2C9 (cytochrome P450 family 2 subfamily C polypeptide 9); ADRA1A (adrenergic alpha-1A- receptor); CD14 (CD14 molecule); IL4R (interleukin 4 receptor); ITPR3 (inositol 1 (4 (5-triphosphate receptor type 3); IL15 (interleukin 15); MECP2 (methyl CpG binding protein 2 (Rett syndrome)); ANXA1 (annexin A1); PRKAG1 (protein kinase AMP-activated gamma 1 non-catalytic subunit); DCN (decorin); MYB (v-myb myeloblastosis viral oncogene homolog (avian)); AVPR1A (arginine vasopressin receptor 1A); HLA-DQB1 (major histocompatibility complex class II DQ beta 1); NEFL (neurofilament light polypeptide); SCNN1B (sodium channel nonvoltage-gated 1 beta); CACNA1H (calcium channel voltage-dependent T type alpha 1H subunit); IFNAR1 (interferon (alpha beta and omega) receptor 1); PDE4D (phosphodiesterase 4D cAMP-specific (phosphodiesterase E3 dunce homolog Drosophila)); HDAC9 (histone deacetylase 9); ABCC1 (ATP-binding cassette sub-family C (CFTR/MRP) member 1); PRDX5 (peroxiredoxin 5); EPHX2 (epoxide hydrolase 2 cytoplasmic); VCAM1 (vascular cell adhesion molecule 1); PRKAG2 (protein kinase AMP-activated gamma 2 non-catalytic subunit); ADCY2 (adenylate cyclase 2 (brain)); HTR1 B (5-hydroxytryptamine (serotonin) receptor 1 B); ADCY9 (adenylate cyclase 9); HLA-A (major histocompatibility complex class I A); SLC1A3 (solute carrier family 1 (glial high affinity glutamate transporter) member 3); HLA-B (major histocompatibility complex class I B); ITGA2 (integrin alpha 2 (CD49B alpha 2 subunit of VLA-2 receptor)); GABRA2 (gamma-aminobutyric acid (GABA) A receptor alpha 2); IL2RB (interleukin 2 receptor beta); GLRB (glycine receptor beta); SOCS3 (suppressor of cytokine signaling 3); CSNK2B (casein kinase 2 beta polypeptide); KCNK3 (potassium channel subfamily K member 3); KCNQ2 (potassium voltage-gated channel KQT-like subfamily member 2); DPYSL2 (dihydropyrimidinase-like 2); CYP2J2 (cytochrome P450 family 2 subfamily J polypeptide 2); DRD4 (dopamine receptor D4); PRKG1 (protein kinase cGMP-dependent type I); TNFSF11 (tumor necrosis factor (ligand) superfamily member 11); IFNAR2 (interferon (alpha beta and omega) receptor 2); EIF4EBP1 (eukaryotic translation initiation factor 4E binding protein 1); EIF4G1 (eukaryotic translation initiation factor 4 gamma 1); EIF4G3 (eukaryotic translation initiation factor 4 gamma 3); SCNN1G (sodium channel nonvoltage-gated 1 gamma); SERPING1 (serpin peptidase inhibitor clade G (C1 inhibitor) member 1); PABPN1 (poly(A) binding protein nuclear 1); CAST (calpastatin); CTSC (cathepsin C); CTGF (connective tissue growth factor); CHRNB2 (cholinergic receptor nicotinic beta 2 (neuronal)); ADCY3 (adenylate cyclase 3); ADCY7 (adenylate cyclase 7); ADRA1 D (adrenergic alpha-1 D- receptor); CHRM2 (cholinergic receptor muscarinic 2); DHFR (dihydrofolate reductase); MC2R (melanocortin 2 receptor (adrenocorticotropic hormone)); THBD (thrombomodulin); IL7 (interleukin 7); IL18 (interleukin 18 (interferon-gamma-inducing factor)); SIRT1 (sirtuin (silent mating type information regulation 2 homolog) 1 (S. cerevisiae)); GRIA4 (glutamate receptor ionotrophic AMPA4); CSNK1 E (casein kinase 1 epsilon); CPE (carboxypeptidase E); PRSS1 (protease serine 1 (trypsin 1)); GOT2 (glutamic-oxaloacetic transaminase 2 mitochondrial (aspartate aminotransferase 2)); GABRB1 (gamma-aminobutyric acid (GABA) A receptor beta 1); ALOX12 (arachidonate 12-lipoxygenase); CCL11 (chemokine (C-C motif) ligand 11); HLA-DRB1 (major histocompatibility complex class II DR beta 1); RBL2 (retinoblastoma-like 2 (p130)); AGER (advanced glycosylation end product-specific receptor); LAMP1 (lysosomal-associated membrane protein 1); MAPKAPK2 (mitogen-activated protein kinase-activated protein kinase 2); LTA (lymphotoxin alpha (TNF superfamily member 1)); CYP4A11 (cytochrome P450 family 4 subfamily A polypeptide 11); MAOB (monoamine oxidase B); TPH1 (tryptophan hydroxylase 1); SPARC (secreted protein acidic cysteine-rich (osteonectin)); PIK3R4 (phosphoinositide-3-kinase regulatory subunit 4); CYP17A1 (cytochrome P450 family 17 subfamily A polypeptide 1); CD63 (CD63 molecule); CLCN1 (chloride channel 1 skeletal muscle); NFE2L2 (nuclear factor (erythroid-derived 2)-like 2); TNFRSF11A (tumor necrosis factor receptor superfamily member 11a NFKB activator); CRHR2 (corticotropin releasing hormone receptor 2); COPE (coatomer protein complex subunit epsilon); CYP4F2 (cytochrome P450 family 4 subfamily F polypeptide 2); APOB (apolipoprotein B (including Ag(x) antigen)); GFRA1 (GDNF family receptor alpha 1); HMBS (hydroxymethylbilane synthase); F5 (coagulation factor V (proaccelerin labile factor)); TPO (thyroid peroxidase); AMPH (amphiphysin); PTGER2 (prostaglandin E receptor 2 (subtype EP2) 53kDa); PKLR (pyruvate kinase liver and RBC); SMPD1 (sphingomyelin phosphodiesterase 1 acid lysosomal); PLA2G4A (phospholipase A2 group IVA (cytosolic calcium-dependent)); JUNB (jun B proto-oncogene); GSN (gelsolin); PLCE1 (phospholipase C epsilon 1); PSMB8 (proteasome (prosome macropain) subunit beta type 8 (large multifunctional peptidase 7)); CYCS (cytochrome c somatic); KCNK1 (potassium channel subfamily K member 1); PGF (placental growth factor); IL1ORA (interleukin 10 receptor alpha); CHRM1 (cholinergic receptor muscarinic 1); IL12RB1 (interleukin 12 receptor beta 1); CHGA (chromogranin A (parathyroid secretory protein 1)); GABRE (gamma-aminobutyric acid (GABA) A receptor epsilon); GJA4 (gap junction protein alpha 4 37 kDa); ALAD (aminolevulinate delta-dehydratase); GLRA2 (glycine receptor alpha 2); ITPR2 (inositol 1 (4 (5-triphosphate receptor type 2); MPZ (myelin protein zero); AQP1 (aquaporin 1 (Colton blood group)); MYBPC3 (myosin binding protein C cardiac); CPT2 (carnitine palmitoyltransferase 2); STAR (steroidogenic acute regulatory protein); GLB1 (galactosidase beta 1); SCN8A (sodium channel voltage gated type VIII alpha subunit); LGALS1 (lectin galactoside-binding soluble 1); PCSK1 (proprotein convertase subtilisin/kexin type 1); IKBKAP (inhibitor of kappa light polypeptide gene enhancer in B-cells kinase complex-associated protein); REST (RE1-silencing transcription factor); OXTR (oxytocin receptor); UGT2B7 (UDP glucuronosyltransferase 2 family polypeptide B7); LTF (lactotransferrin); TYRP1 (tyrosinase-related protein 1); RBL1 (retinoblastoma-like 1 (p107)); TCAP (titin-cap (telethonin)); KCNJ1 (potassium inwardly-rectifying channel subfamily J member 1); KCNN3 (potassium intermediate/small conductance calcium-activated channel subfamily N member 3); PSMC1 (proteasome (prosome macropain) 26S subunit ATPase 1); RELN (reelin); MYH14 (myosin heavy chain 14 non-muscle); ADCY4 (adenylate cyclase 4); MMP10 (matrix metallopeptidase 10 (stromelysin 2)); FXN (frataxin); ATF4 (activating transcription factor 4 (tax-responsive enhancer element B67)); NOG (noggin); PPDX (protoporphyrinogen oxidase); TNNC1 (troponin C type 1 (slow)); HRH2 (histamine receptor H2); PLA2G4C (phospholipase A2 group IVC (cytosolic calcium-independent)); NR3C2 (nuclear receptor subfamily 3 group C member 2); AMPD1 (adenosine monophosphate deaminase 1); FKBP4 (FK506 binding protein 4 59kDa); MBD2 (methyl-CpG binding domain protein 2); NRG1 (neuregulin 1); MBL2 (mannose-binding lectin (protein C) 2 soluble (opsonic defect)); AGA (aspartylglucosaminidase); SP1 (Sp1 transcription factor); SCN3A (sodium channel voltage-gated type III alpha subunit); FABP2 (fatty acid binding protein 2 intestinal); PABPC1 (poly(A) binding protein cytoplasmic 1); ACCN2 (amiloride-sensitive cation channel 2 neuronal); ACTC1 (actin alpha cardiac muscle 1); ACP5 (acid phosphatase 5 tartrate resistant); EIF4B (eukaryotic translation initiation factor 4B); EIF4EBP2 (eukaryotic translation initiation factor 4E binding protein 2); EIF4A1 (eukaryotic translation initiation factor 4A1); CAMK4 (calcium/calmodulin-dependent protein kinase IV); CACNB3 (calcium channel voltage-dependent beta 3 subunit); CAV3 (caveolin 3); CA6 (carbonic anhydrase VI); ALOX12B (arachidonate 12-lipoxygenase 12R type); CCL17 (chemokine (C-C motif) ligand 17); CCL22 (chemokine (C-C motif) ligand 22); MMP20 (matrix metallopeptidase 20); GAP43 (growth associated protein 43); ALOX5AP (arachidonate 5-lipoxygenase-activating protein); ANTXR2 (anthrax toxin receptor 2); HGD (homogentisate 1 (2-dioxygenase); SELE (selectin E); MYLK2 (myosin light chain kinase 2); VEGFA (vascular endothelial growth factor A); PRX (periaxin); IL1ORB (interleukin 10 receptor beta); HAS1 (hyaluronan synthase 1); GTF2IRD1 (GTF2I repeat domain containing 1); IL16 (interleukin 16 (lymphocyte chemoattractant factor)); GRIP1 (glutamate receptor interacting protein 1); PHKA1 (phosphorylase kinase alpha 1 (muscle)); FOXP3 (forkhead box P3); SFTPC (surfactant protein C); PDIA3 (protein disulfide isomerase family A member 3); SRM (spermidine synthase); MARCKS (myristoylated alanine-rich protein kinase C substrate); RAPGEF3 (Rap guanine nucleotide exchange factor (GEF) 3); RAGE (renal tumor antigen); MRC1 (mannose receptor C type 1); SPINK1 (serine peptidase inhibitor Kazal type 1); CYP4F3 (cytochrome P450 family 4 subfamily F polypeptide 3); LPIN1 (lipin 1); TREX1 (three prime repair exonuclease 1); CYSLTR2 (cysteinyl leukotriene receptor 2); PTX3 (pentraxin 3 long); PTGES2 (prostaglandin E synthase 2); ASAH1 (N-acylsphingosine amidohydrolase (acid ceramidase) 1); H2AFZ (H2A histone family member Z); HFE (hemochromatosis); PYGB (phosphorylase glycogen; brain); NR2F6 (nuclear receptor subfamily 2 group F member 6); CYP3A7 (cytochrome P450 family 3 subfamily A polypeptide 7); RAB6A (RAB6A member RAS oncogene family); F2RL3 (coagulation factor II (thrombin) receptor-like 3); RGS4 (regulator of G-protein signaling 4); SCNN1 D (sodium channel nonvoltage-gated 1 delta); SCN1 B (sodium channel voltage-gated type I beta); SCN2A (sodium channel voltage-gated type II alpha subunit); CALCRL (calcitonin receptor-like); CALB1 (calbindin 1 28kDa); CACNG2 (calcium channel voltage-dependent gamma subunit 2); TACR2 (tachykinin receptor 2); GPC3 (glypican 3); GALNT3 (UDP-N-acetyl-alpha-D-galactosamine:polypeptide N-acetylgalactosaminyltransferase 3 (GalNAc-T3)); CXCL10 (chemokine (C-X-C motif) ligand 10); ANKH (ankylosis progressive homolog (mouse)); PRKD1 (protein kinase D1); KCNN4 (potassium intermediate/small conductance calcium-activated channel subfamily N member 4); TGM1 (transglutaminase 1 (K polypeptide epidermal type I protein-glutamine-gamma-glutamyltransferase)); SLC26A2 (solute carrier family 26 (sulfate transporter) member 2); MTNR1A (melatonin receptor 1A); MIPEP (mitochondrial intermediate peptidase); SI (sucrase-isomaltase (alpha-glucosidase)); RHAG (Rh-associated glycoprotein); SLC12A3 (solute carrier family 12 (sodium/chloride transporters) member 3); RNASE1 (ribonuclease RNase A family 1 (pancreatic)); ELANE (elastase neutrophil expressed); GPC6 (glypican 6); ENPP2 (ectonucleotide pyrophosphatase/phosphodiesterase 2); SCN3B (sodium channel voltage-gated type III beta); CALB2 (calbindin 2); CTSA (cathepsin A); EIF2AK1 (eukaryotic translation initiation factor 2-alpha kinase 1); TMSB4X (thymosin beta 4 X-linked); LPO (lactoperoxidase); NDN (necdin homolog (mouse)); PICK1 (protein interacting with PRKCA 1); PLCD4 (phospholipase C delta 4); CLDN3 (claudin 3); HCN1 (hyperpolarization activated cyclic nucleotide-gated potassium channel 1); MATN3 (matrilin 3); COL9A3 (collagen type IX alpha 3); BTG1 (B-cell translocation gene 1 anti-proliferative); LCN1 (lipocalin 1 (tear prealbumin)); FDX1 (ferredoxin 1); UTRN (utrophin); FMOD (fibromodulin); PDE4A (phosphodiesterase 4A cAMP-specific (phosphodiesterase E2 dunce homolog Drosophila)); RRBP1 (ribosome binding protein 1 homolog 180kDa (dog)); MLYCD (malonyl-CoA decarboxylase); ANXA3 (annexin A3); PRKD3 (protein kinase D3); GHRL (ghrelin/obestatin prepropeptide); GDF15 (growth differentiation factor 15); BCL11A (B-cell CLL/lymphoma 11A (zinc finger protein)); CSRP3 (cysteine and glycine-rich protein 3 (cardiac LIM protein)); CXCL2 (chemokine (C-X-C motif) ligand 2); TOMM40 (translocase of outer mitochondrial membrane 40 homolog (yeast)); KCNK6 (potassium channel subfamily K member 6); KCNN2 (potassium intermediate/small conductance calcium-activated channel subfamily N member 2); SLC6Al2 (solute carrier family 6 (neurotransmitter transporter betaine/GABA) member 12); ALOXE3 (arachidonate lipoxygenase 3); SOST (sclerosteosis); PRLHR (prolactin releasing hormone receptor); TIMM44 (translocase of inner mitochondrial membrane 44 homolog (yeast)); KCNN1 (potassium intermediate/small conductance calcium-activated channel subfamily N member 1); CHRNA9 (cholinergic receptor nicotinic alpha 9); GPC5 (glypican 5); GPR37 (G protein-coupled receptor 37 (endothelin receptor type B-like)); NKX2-1 (NK2 homeobox 1); HMMR (hyaluronan-mediated motility receptor (RHAMM)); PKHD1 (polycystic kidney and hepatic disease 1 (autosomal recessive)); AOC2 (amine oxidase copper containing 2 (retina-specific)); KRT20 (keratin 20); CORIN (corin serine peptidase); AZU1 (azurocidin 1); MAPK6 (mitogen-activated protein kinase 6); PAEP (progestagen-associated endometrial protein); CACNA2D4 (calcium channel voltage-dependent alpha 2/delta subunit 4); EIF3A (eukaryotic translation initiation factor 3 subunit A); BTG2 (BTG family member 2); P2RY14 (purinergic receptor P2Y G-protein coupled 14); PDLIM7 (PDZ and LIM domain 7 (enigma)); CACNA2D3 (calcium channel voltage-dependent alpha 2/delta subunit 3); LAMP3 (lysosomal-associated membrane protein 3); PLCL2 (phospholipase C-like 2); NOSIP (nitric oxide synthase interacting protein); CRHBP (corticotropin releasing hormone binding protein); KLK5 (kallikrein-related peptidase 5); ADAM2 (ADAM metallopeptidase domain 2); SIRPA (signal-regulatory protein alpha); PMPCB (peptidase (mitochondrial processing) beta); GPC4 (glypican 4); MYH6 (myosin heavy chain 6 cardiac muscle alpha); CXCL9 (chemokine (C-X-C motif) ligand 9); KCNK5 (potassium channel subfamily K member 5); KCNK10 (potassium channel subfamily K member 10); NMU (neuromedin U); SCN4B (sodium channel voltage-gated type IV beta); CAMK1 D (calcium/calmodulin-dependent protein kinase ID); COL8A2 (collagen type VIII alpha 2); RAB11FIP1 (RAB11 family interacting protein 1 (class I)); NDOR1 (NADPH dependent diflavin oxidoreductase 1); ZNF318 (zinc finger protein 318); P2RX2 (purinergic receptor P2X ligand-gated ion channel 2); UGT1A6 (UDP glucuronosyltransferase 1 family polypeptide A6); LEMD3 (LEM domain containing 3); UGT1A1 (UDP glucuronosyltransferase 1 family polypeptide Al); PDLIM3 (PDZ and LIM domain 3); KCTD12 (potassium channel tetramerisation domain containing 12); KCNK9 (potassium channel subfamily K member 9); DSE (dermatan sulfate epimerase); DSPP (dentin sialophosphoprotein); KCNT2 (potassium channel subfamily T member 2); NMUR2 (neuromedin U receptor 2); CHST6 (carbohydrate (N-acetylglucosamine 6-O) sulfotransferase 6); CCL28 (chemokine (C-C motif) ligand 28); SLPI (secretory leukocyte peptidase inhibitor); CCL1 (chemokine (C-C motif) ligand 1); KCNK15 (potassium channel subfamily K member 15); KCTD15 (potassium channel tetramerisation domain containing 15); ANKRD1 (ankyrin repeat domain 1 (cardiac muscle)); SIGMAR1 (sigma non-opioid intracellular receptor 1); SLCO2A1 (solute carrier organic anion transporter family member 2A1); MUC16 (mucin 16 cell surface associated); CNTNAP1 (contactin associated protein 1); LGR6 (leucine-rich repeat-containing G protein-coupled receptor 6); ASPN (asporin); PLCH2 (phospholipase C eta 2); PLCL1 (phospholipase C-like 1); AGFG1 (ArfGAP with FG repeats 1); HOXB8 (homeobox B8); KCNK12 (potassium channel subfamily K member 12); KCNK4 (potassium channel subfamily K member 4); KCNRG (potassium channel regulator); KCTD13 (potassium channel tetramerisation domain containing 13); KCNT1 (potassium channel subfamily T member 1); RNF19A (ring finger protein 19A); CIAPIN1 (cytokine induced apoptosis inhibitor 1); TNS3 (tensin 3); AMELX (amelogenin X-linked); CRBN (cereblon); MLN (motilin); CXCR1 (chemokine (C-X-C motif) receptor 1); NPBWR2 (neuropeptides B/W receptor 2); KCMF1 (potassium channel modulatory factor 1); KCNK7 (potassium channel subfamily K member 7); KCNV1 (potassium channel subfamily V member 1); KCTD5 (potassium channel tetramerisation domain containing 5); KCNV2 (potassium channel subfamily V member 2); KCNK13 (potassium channel subfamily K member 13); ERAP2 (endoplasmic reticulum aminopeptidase 2); KCTD2 (potassium channel tetramerisation domain containing 2); KCTD3 (potassium channel tetramerisation domain containing 3); KCNK17 (potassium channel subfamily K member 17); KCTD10 (potassium channel tetramerisation domain containing 10); KCTD7 (potassium channel tetramerisation domain containing 7); SCT (secretin); NGDN (neuroguidin EIF4E binding protein); MLNR (motilin receptor); MPZL2 (myelin protein zero-like 2); PROL1 (proline rich lacrimal 1); KCNK16 (potassium channel subfamily K member 16); KCTD9 (potassium channel tetramerisation domain containing 9); KCTD11 (potassium channel tetramerisation domain containing 11); KCTD8 (potassium channel tetramerisation domain containing 8); KCTD4 (potassium channel tetramerisation domain containing 4); KCTD6 (potassium channel tetramerisation domain containing 6); KCTD1 (potassium channel tetramerisation domain containing 1); NPVF (neuropeptide VF precursor); MAGIX (MAGI family member X-linked); MRGPRX4 (MAS-related GPR member X4); MRGPRD (MAS-related GPR member D); TET2 (tet oncogene family member 2); KCTD14 (potassium channel tetramerisation domain containing 14); GLYATL1 (glycine-N-acyltransferase-like 1); ZNF493 (zinc finger protein 493); ZNF429 (zinc finger protein 429); MRGPRE (MAS-related GPR member E); SUN2 (Sad1 and UNC84 domain containing 2); AMTN (amelotin); MRGPRF (MAS-related GPR member F); CDK20 (cyclin-dependent kinase 20); KCNU1 (potassium channel subfamily U member 1); GATS (GATS stromal antigen 3 opposite strand); GLRA4 (glycine receptor alpha 4); IGHE (immunoglobulin heavy constant epsilon); DRGX (dorsal root ganglia homeobox); MRGPRG (MAS-related GPR member G); LOC729977 (hypothetical LOC729977); MT-TK (mitochondrially encoded tRNA lysine); LOC400680 (hypothetical gene supported by AK097381; BC040866); COP (clathrin-ordered protein); IGES (immunoglobulin E concentration serum); MGS (Mungen syndrome); TRNAS-AGA (transfer RNA serine (anticodon AGA)); and LOC100132258 (similar to secretory carrier membrane protein 2).

Non-limiting examples of taste-related genes include TAS2R38 (taste receptor, type 2, member 38); TAS1 R1 (taste receptor, type 1, member 1); TAS2R3 (taste receptor, type 2, member 3); TAS2R5 (taste receptor, type 2, member 5); TAS2R1 (taste receptor, type 2, member 1); TAS2R16 (taste receptor, type 2, member 16); TAS2R4 (taste receptor, type 2, member 4); TAS2R14 (taste receptor, type 2, member 14); TAS2R10 (taste receptor, type 2, member 10); TAS2R7 (taste receptor, type 2, member 7); TAS2R13 (taste receptor, type 2, member 13); TAS2R9 (taste receptor, type 2, member 9); TAS2R8 (taste receptor, type 2, member 8); TAS1 R3 (taste receptor, type 1, member 3); TAS2R31 (taste receptor, type 2, member 31); TAS1 R2 (taste receptor, type 1, member 2); TAS2R43 (taste receptor, type 2, member 43); TAS2R50 (taste receptor, type 2, member 50); TAS2R46 (taste receptor, type 2, member 46); TAS2R30 (taste receptor, type 2, member 30); TAS2R42 (taste receptor, type 2, member 42); PLCB2 (phospholipase C, beta 2); TAS2R20 (taste receptor, type 2, member 20); TAS2R19 (taste receptor, type 2, member 19); GNG13 ((guanine nucleotide binding protein (G protein)), gamma 13); TAS2R12 (taste receptor, type 2, member 12 pseudogene); GNAT1 (guanine nucleotide binding protein (G protein), alpha transducing activity polypeptide 1); TAS2R41 (taste receptor, type 2, member 41); TAS2R60 (taste receptor, type 2, member 60); TAS2R40 (taste receptor, type 2, member 40); TAS2R39 (taste receptor, type 2, member 39); GCG (glucagon); TAS2R18 (taste receptor, type 2, member 18 pseudogene); GRM4 (glutamate receptor, metabotropic 4); LCN1 (lipocalin 1 (tear prealbumin)); TRPV1 (transient receptor potential cation channel, subfamily V, member 1); ACCN1 (amiloride-sensitive cation channel 1, neuronal); TAS2R45 (taste receptor, type 2, member 45); TAS2R15 (taste receptor, type 2, member 15 pseudogene); FOS (murine osteosarcoma viral oncogene homolog); SLC9A1 (solute carrier family 9 (sodium/hydrogen exchanger), member 1); INS (insulin); ACCN5 (amiloride-sensitive cation channel 5, intestinal); TAS2R2 (taste receptor, type 2, member 2 pseudogene); GRM7 (glutamate receptor, metabotropic 7); NPY (neuropeptide Y); LEP (leptin); CASR (calcium-sensing receptor); GNAZ (guanine nucleotide binding protein (G protein), alpha z polypeptide); CIB1 (calcium and integrin binding 1 (calmyrin)); ADCY10 (adenylate cyclase 10 (soluble)); LEPR (leptin receptor); DRD1 (dopamine receptor D1); LGR6 (leucine-rich repeat-containing G protein-coupled receptor 6); GRM8 (glutamate receptor, metabotropic 8); GRM6 (glutamate receptor, metabotropic 6); GLP1 R (glucagon-like peptide 1 receptor); AGER (advanced glycosylation end product-specific receptor); SLC2A2 (solute carrier family 2 (facilitated glucose transporter), member 2); GIP (gastric inhibitory polypeptide); REN (rennin); PDYN (prodynorphin); RRBP1 (ribosome binding protein 1 homolog 180kDa (dog)); SLC15A1 (solute carrier family 15 (oligopeptide transporter), member 1); OXT (oxytocin, prepropeptide); IL411 (interleukin 4 induced 1); VN1R17P (vomeronasal 1 receptor 17 pseudogene); TAS2R62P (taste receptor, type 2, member 62, pseudogene); TAS2R64P (taste receptor, type 2, member 64 pseudogene); TAS2R63P (taste receptor, type 2, member 63 pseudogene); PS5 (bitter taste receptor pseudogene PS5); PS3 (bitter taste receptor PS3); PS7 (bitter taste receptor Ps7 pseudogene); C6orf15 (chromosome 6 open reading frame 15); TAS2R6 (taste receptor, type 2, member 6); TAS2R22 (taste receptor, type 2, member 22); TAS2R33 (taste receptor, type 2, member 33); TAS2R37 (taste receptor, type 2, member 37); TAS2R36 (taste receptor, type 2, member 36); GNAT3 (guanine nucleotide binding protein, alpha transducing 3); TRPM5 (transient receptor potential cation channel, subfamily M, member 5); TRPM7 (transient receptor potential cation channel, subfamily M, member 7); GNB1 (guanine nucleotide binding protein (G protein), beta polypeptide 1); ITPR3 (inositol 1,4,5-triphosphate receptor, type 3); ACE (angiotensin I converting enzyme (peptidyl-dipeptidase A) 1); ENO2 (enolase 2 (gamma, neuronal)); CALCA (calcitonin-related polypeptide alpha); CCK (cholecystokinin); RTP3 (receptor (chemosensory) transporter protein 3); PL-5283 (PL-5283 protein); PRKCG (protein kinase C, gamma); KCNQ1 (potassium voltage-gated channel, KQT-like subfamily, member 1); BDNF (brain-derived neurotrophic factor); SCNN1A (sodium channel, nonvoltage-gated 1 alpha); GNB3 (guanine nucleotide binding protein (G protein), beta polypeptide 3); SCNN1 B (sodium channel, nonvoltage-gated 1, beta); SCNN1G (sodium channel, nonvoltage-gated 1, gamma); GNB4 (guanine nucleotide binding protein (G protein), beta polypeptide 4); PDE1A (phosphodiesterase 1A, calmodulin-dependent); DMBT1 (deleted in malignant brain tumors 1); PDE3B (phosphodiesterase 3B, cGMP-inhibited); PDE1C (phosphodiesterase 1C, calmodulin-dependent 70kDa); PRKCA (protein kinase C, alpha); NTRK3 (neurotrophic tyrosine kinase, receptor, type 3); NTRK2 (neurotrophic tyrosine kinase, receptor, type 2); PRKCQ (protein kinase C, theta); PRKACA (protein kinase, cAMP-dependent, catalytic, alpha); CCKBR (cholecystokinin B receptor); PRKCZ (protein kinase C, zeta); TH (tyrosine hydroxylase); NGFR (nerve growth factor receptor (TNFR superfamily, member 16)); DRD2 (dopamine receptor D2); NOS1 (nitric oxide synthase 1 (neuronal)); PRKCE (protein kinase C, epsilon); PRKCH (protein kinase C, eta); PRKCD (protein kinase C, delta); ABCB1 (ATP-binding cassette, sub-family B (MDR/TAP), member 1); MAPK1 (mitogen-activated protein kinase 1); PLCB3 (phospholipase C, beta 3 (phosphatidylinositol-specific)); ADCY8 (adenylate cyclase 8 (brain)); ADRBK2 (adrenergic, beta, receptor kinase 2); PRKACB (protein kinase, cAMP-dependent, catalytic, beta); PRKCI (protein kinase C, iota); CCKAR (cholecystokinin A receptor); KCNK3 (potassium channel, subfamily K, member 3); PLCB1 (phospholipase C, beta 1 (phosphoinositide-specific)); ADCY3 (adenylate cyclase 3); NTF3 (neurotrophin 3); PLCB4 (phospholipase C, beta 4); GNB5 (guanine nucleotide binding protein (G protein), beta 5); GNAL (guanine nucleotide binding protein (G protein), alpha activating activity polypeptide, olfactory type); GNB2 (guanine nucleotide binding protein (G protein), beta polypeptide 2); KCNK1 (potassium channel, subfamily K, member 1); HTR1A (5-hydroxytryptamine (serotonin) receptor 1A); CNGA3 (cyclic nucleotide gated channel alpha 3); PRKACG (protein kinase, cAMP-dependent, catalytic, gamma); PRKCB (protein kinase C, beta); RBP4 (retinol binding protein 4, plasma); GRP (gastrin-releasing peptide); PDE3A (phosphodiesterase 3A, cGMP-inhibited); KRT14 (keratin 14); SCNN1D (sodium channel, nonvoltage-gated 1, delta); PRKD1 (protein kinase D1); PDE1B (phosphodiesterase 1B, calmodulin-dependent); PDE2A (phosphodiesterase 2A, cGMP-stimulated); PRKD3 (protein kinase D3); SST (somatostatin); KCNK6 (potassium channel, subfamily K, member 6); KCNK2 (potassium channel, subfamily K, member 2); NTF4 (neurotrophin 4); GNG3 (guanine nucleotide binding protein (G protein), gamma 3); RNH1 (ribonuclease/angiogenin inhibitor 1); KCNK5 (potassium channel, subfamily K, member 5); KCNK10 (potassium channel, subfamily K, member 10); P2RX2 (purinergic receptor P2X, ligand-gated ion channel, 2); KCTD12 (potassium channel tetramerisation domain containing 12); KCNK9 (potassium channel, subfamily K, member 9); KCNT2 (potassium channel, subfamily T, member 2); KCNK15 (potassium channel, subfamily K, member 15); KCTD15 (potassium channel tetramerisation domain containing 15); KCNK12 (potassium channel, subfamily K, member 12); KCNK4 (potassium channel, subfamily K, member 4); KCNRG (potassium channel regulator); KCTD13 (potassium channel tetramerisation domain containing 13); KCNT1 (potassium channel, subfamily T, member 1); KCMF1 (potassium channel modulatory factor 1); KCNK7 (potassium channel, subfamily K, member 7); KCNV1 (potassium channel, subfamily V, member 1); KCTD5 (potassium channel tetramerisation domain containing 5); KCNV2 (potassium channel, subfamily V, member 2); KCNK13 (potassium channel, subfamily K, member 13); KCTD2 (potassium channel tetramerisation domain containing 2); KCTD3 (potassium channel tetramerisation domain containing 3); KCNK17 (potassium channel, subfamily K, member 17); KCTD10 (potassium channel tetramerisation domain containing 10); KCTD7 (potassium channel tetramerisation domain containing 7); KCNK16 (potassium channel, subfamily K, member 16); KCTD9 (potassium channel tetramerisation domain containing 9); KCTD11 (potassium channel tetramerisation domain containing 11); KCTD8 (potassium channel tetramerisation domain containing 8); KCTD4 (potassium channel tetramerisation domain containing 4); KCTD6 (potassium channel tetramerisation domain containing 6); KCTD1 (potassium channel tetramerisation domain containing 1); KCTD14 (potassium channel tetramerisation domain containing 14); RTP4 (receptor (chemosensory) transporter protein 4); KCNU1 (potassium channel, subfamily U, member 1); LOC730036 (hypothetical LOC730036); RPS6KA3 (ribosomal protein S6 kinase, 90kDa, polypeptide 3); MAPT (microtubule-associated protein tau); CHEK2 (CHK2 checkpoint homolog (S. pombe)); FYN (FYN oncogene related to SRC, FGR, YES); APP (amyloid beta (A4) precursor protein); PTEN (phosphatase and tensin homolog); SOD1 (superoxide dismutase 1, soluble); CSTB (cystatin B (stefin B)); SHH (sonic hedgehog homolog (Drosophila)); AKR1 B1 (aldo-keto reductase family 1, member B1 (aldose reductase)); COMT (catechol-O-methyltransferase); S100B (S100 calcium binding protein B); PTK2B (PTK2B protein tyrosine kinase 2 beta); PLCG2 (phospholipase C, gamma 2 (phosphatidylinositol-specific)); PSEN1 (presenilin 1); SLC6A3 (solute carrier family 6 (neurotransmitter transporter, dopamine), member 3); PAX6 (paired box 6); MMP1 (matrix metallopeptidase 1 (interstitial collagenase)); CACNA1A (calcium channel, voltage-dependent, P/Q type, alpha 1A subunit); CASP9 (caspase 9, apoptosis-related cysteine peptidase); PRKAR1A (protein kinase, cAMP-dependent, regulatory, type I, alpha (tissue specific extinguisher 1)); MMP3 (matrix metallopeptidase 3 (stromelysin 1, progelatinase)); ADCY6 (adenylate cyclase 6); CASP3 (caspase 3, apoptosis-related cysteine peptidase); GNAS (GNAS complex locus); MMP9 (matrix metallopeptidase 9 (gelatinase B, 92kDa gelatinase, 92kDa type IV collagenase)); NOTCH2 (Notch homolog 2 (Drosophila)); CREB1 (cAMP responsive element binding protein 1); SNCA (synuclein, alpha (non A4 component of amyloid precursor)); OPRM1 (opioid receptor, mu 1); CALM1 (calmodulin 1 (phosphorylase kinase, delta)); PLCG1 (phospholipase C, gamma 1); BRCA1 (breast cancer 1, early onset); APOE (apolipoprotein E); DBH (dopamine beta-hydroxylase (dopamine beta-monooxygenase)); PTGS2 (prostaglandin-endoperoxide synthase 2 (prostaglandin G/H synthase and cyclooxygenase)); ADRBK1 (adrenergic, beta, receptor kinase 1); ITGB4 (integrin, beta 4); NLGN3 (neuroligin 3); CD36 (CD36 molecule (thrombospondin receptor)); EEF2 (eukaryotic translation elongation factor 2); OPRD1 (opioid receptor, delta 1); HSPG2 (heparan sulfate proteoglycan 2); GAD1 (glutamate decarboxylase 1 (brain, 67kDa)); ANXA1 (annexin A1); PRKAR2A (protein kinase, cAMP-dependent, regulatory, type II, alpha); HHEX (hematopoietically expressed homeobox); GRM1 (glutamate receptor, metabotropic 1); NPR1 (natriuretic peptide receptor A/guanylate cyclase A (atrionatriuretic), peptide receptor A); SYP (synaptophysin); CALM3 (calmodulin 3 (phosphorylase kinase, delta)); PRKAR2B (protein kinase, cAMP-dependent, regulatory, type II, beta); ADCY2 (adenylate cyclase 2 (brain)); SLC1A3 (solute carrier family 1 (glial high affinity glutamate transporter), member 3); GABBR1 (gamma-aminobutyric acid (GABA) B receptor, 1); PTPRS (protein tyrosine phosphatase, receptor type, S); KNG1 (kininogen 1); DDC (dopa decarboxylase (aromatic L-amino acid decarboxylase)); GNAQ (guanine nucleotide binding protein (G protein), q polypeptide); E2F4 (E2F transcription factor 4, p107/p130-binding); DRD4 (dopamine receptor D4); MAOA (monoamine oxidase A); CALM2 (calmodulin 2 (phosphorylase kinase, delta)); CHRNB2 (cholinergic receptor, nicotinic, beta 2 (neuronal)); GRK5 (G protein-coupled receptor kinase 5); PRLR (prolactin receptor); ID2 (inhibitor of DNA binding 2, dominant negative helix-loop-helix protein); TPH1 (tryptophan hydroxylase 1); PLCD1 (phospholipase C, delta 1); GNA11 (guanine nucleotide binding protein (G protein), alpha 11 (Gq class)); GNAl2 (guanine nucleotide binding protein (G protein) alpha 12); CRH (corticotropin releasing hormone); GNRH1 (gonadotropin-releasing hormone 1 (luteinizing-releasing hormone)); S100A8 (S100 calcium binding protein A8); CYCS (cytochrome c, somatic); KCNB1 (potassium voltage-gated channel, Shab-related subfamily, member 1); DST (dystonin); ADCY1 (adenylate cyclase 1 (brain)); CHGA (chromogranin A (parathyroid secretory protein 1)); HTR3A (5-hydroxytryptamine (serotonin) receptor 3A); GAL (galanin prepropeptide); TACR3 (tachykinin receptor 3); ALDH7A1 (aldehyde dehydrogenase 7 family, member Al); PRKAR1 B (protein kinase, cAMP-dependent, regulatory, type I, beta); AQP5 (aquaporin 5); AQP2 (aquaporin 2 (collecting duct)); AQP1 (aquaporin 1 (Colton blood group)); GLI3 (GLI family zinc finger 3); POU2F1 (POU class 2 homeobox 1); OTX2 (orthodenticle homeobox 2); TTR (transthyretin); CACNA1 B (calcium channel, voltage-dependent, N type, alpha 1B subunit); IKBKAP (inhibitor of kappa light polypeptide gene enhancer in B-cells, kinase complex-associated protein); RHO (rhodopsin); UGT2B7 (UDP glucuronosyltransferase 2 family, polypeptide B7); LCT (lactase); TCOF1 (Treacher Collins-Franceschetti syndrome 1); KCNJ1 (potassium inwardly-rectifying channel, subfamily J, member 1); VIP (vasoactive intestinal peptide); AQP3 (aquaporin 3 (Gill blood group)); TAC1 (tachykinin, precursor 1); ADCY4 (adenylate cyclase 4); HP (haptoglobin); ALDH4A1 (aldehyde dehydrogenase 4 family, member A1); GDI1 (GDP dissociation inhibitor 1); SOX2 (SRY (sex determining region Y)-box 2); NOG (noggin); FST (follistatin); NDST1 (N-deacetylase/N-sulfotransferase (heparan glucosaminyl) 1); ABLIM1 (actin binding LIM protein 1); NOS2 (nitric oxide synthase 2, inducible); EIF2B1 (eukaryotic translation initiation factor 2B, subunit 1 alpha, 26kDa); CA6 (carbonic anhydrase VI); DKK1 (dickkopf homolog 1 (Xenopus laevis)); SIX3 (SIX homeobox 3); SIX1 (SIX homeobox 1); HTT (huntingtin); AGRP (agouti related protein homolog (mouse)); NCAM2 (neural cell adhesion molecule 2); BBS4 (Bardet-Biedl syndrome 4); GNA15 (guanine nucleotide binding protein (G protein), alpha 15 (Gq class)); GNA13 (guanine nucleotide binding protein (G protein), alpha 13); ASCL1 (achaete-scute complex homolog 1 (Drosophila)); MGLL (monoglyceride lipase); PLCD3 (phospholipase C, delta 3); CEBPB (CCAAT/enhancer binding protein (C/EBP), beta); BBS1 (Bardet-Biedl syndrome 1); HES1 (hairy and enhancer of split 1, (Drosophila)); GNG2 (guanine nucleotide binding protein (G protein), gamma 2); TPH2 (tryptophan hydroxylase 2); P2RX3 (purinergic receptor P2X, ligand-gated ion channel, 3); AQP7 (aquaporin 7); CNGB1 (cyclic nucleotide gated channel beta 1); GABRR1 (gamma-aminobutyric acid (GABA) receptor, rho 1); GBX2 (gastrulation brain homeobox 2); SLC6A1 (solute carrier family 6 (neurotransmitter transporter, GABA), member 1); PEBP1 (phosphatidylethanolamine binding protein 1); KRT13 (keratin 13); NAV2 (neuron navigator 2); BBS2 (Bardet-Biedl syndrome 2); PLCD4 (phospholipase C, delta 4); CLDN8 (claudin 8); CLDN7 (claudin 7); CISH (cytokine inducible SH2-containing protein); GNGT2 (guanine nucleotide binding protein (G protein), gamma transducing activity polypeptide 2); GNG4 (guanine nucleotide binding protein (G protein), gamma 4); GNA14 (guanine nucleotide binding protein (G protein), alpha 14); UCN (urocortin); PDE4A (phosphodiesterase 4A, cAMP-specific (phosphodiesterase E2 dunce homolog, Drosophila)); MKKS (McKusick-Kaufman syndrome); GAST (gastrin); PRKX (protein kinase, X-linked); CHRD (chordin); PRSS2 (protease, serine, 2 (trypsin 2)); KRT20 (keratin 20); CLDN6 (claudin 6); CLCN4 (chloride channel 4); DLX5 (distal-less homeobox 5); TRPA1 (transient receptor potential cation channel, subfamily A, member 1); TRPM8 (transient receptor potential cation channel, subfamily M, member 8); PLCZ1 (phospholipase C, zeta 1); SLC5A2 (solute carrier family 5 (sodium/glucose cotransporter), member 2); GDF11 (growth differentiation factor 11); BLVRB (biliverdin reductase B (flavin reductase (NADPH))); SCN7A (sodium channel, voltage-gated, type VII, alpha); PANX1 (pannexin 1); IF135 (interferon-induced protein 35); NRAP (nebulin-related anchoring protein); HES5 (hairy and enhancer of split 5 (Drosophila)); GSC (goosecoid homeobox); REEP1 (receptor accessory protein 1); CCL28 (chemokine (C-C motif) ligand 28); GJB4 (gap junction protein, beta 4, 30.3kDa); B3GNT2 (UDP-GlcNAc:betaGal beta-1,3-N-acetylglucosaminyltransferase 2); CNGA2 (cyclic nucleotide gated channel alpha 2); ZNF423 (zinc finger protein 423); HESX1 (HESX homeobox 1); CNGA4 (cyclic nucleotide gated channel alpha 4); GPR158 (G protein-coupled receptor 158); MAGEL2 (MAGE-like 2); UBR3 (ubiquitin protein ligase E3 component n-recognin 3 (putative)); NPTXR (neuronal pentraxin receptor); SLC24A6 (solute carrier family 24 (sodium/potassium/calcium exchanger), member 6); GPRC6A (G protein-coupled receptor, family C, group 6, member A); SLC24A3 (solute carrier family 24 (sodium/potassium/calcium exchanger), member 3); BEST2 (bestrophin 2); OR8D2 (olfactory receptor, family 8, subfamily D, member 2); OR5P2 (olfactory receptor, family 5, subfamily P, member 2); FOXG1 (forkhead box G1); OR8B8 (olfactory receptor, family 8, subfamily B, member 8); OR8D1 (olfactory receptor, family 8, subfamily D, member 1); OR10A5 (olfactory receptor, family 10, subfamily A, member 5); OMP (olfactory marker protein); TFAP2E (transcription factor AP-2 epsilon (activating enhancer binding protein 2, epsilon); OR5P3 (olfactory receptor, family 5, subfamily P, member 3); OR10A4 (olfactory receptor, family 10, subfamily A, member 4); DMRTA1 (DMRT-like family Al); TMEM147 (transmembrane protein 147); OR8A1 (olfactory receptor, family 8, subfamily A, member 1); EBF2 (early B-cell factor 2); PKD1 L3 (polycystic kidney disease 1-like 3); GPR179 (G protein-coupled receptor 179); RTP1 (receptor (chemosensory) transporter protein 1); KLHL35 (kelch-like 35 (Drosophila)); RGS21 (regulator of G-protein signaling 21); RTP2 (receptor (chemosensory) transporter protein 2); ACSM4 (acyl-CoA synthetase medium-chain family member 4); GUCY2E (guanylate cyclase 2E); CYP2G1 P (cytochrome P450, family 2, subfamily G, polypeptide 1 pseudogene); OR7E35P (olfactory receptor, family 7, subfamily E, member 35 pseudogene); and NUDT16P1 (nudix (nucleoside diphosphate linked moiety X-type motif 16, pseudogene 1).

Preferred sensory-related genes may include TRPM7 (transient receptor potential cation channel, subfamily M, member 7); TRPM5 (transient receptor potential cation channel, subfamily M, member 5); TRPC5 (transient receptor potential cation channel subfamily C member 5); TRPC6 (transient receptor potential cation channel subfamily C member 6); TRPC1 (transient receptor potential cation channel subfamily C member 1); CNR1 (cannabinoid receptor 1 (brain)); CNR2 (cannabinoid receptor 2 (macrophage)); ADRBK1 (adrenergic beta receptor kinase 1); TRPA1 (transient receptor potential cation channel subfamily A member 1); POMC (proopiomelanocortin); CALCA (CGRP, calcitonin-related polypeptide alpha); CRF (CRH, corticotrophin releasing factor); PKA such as PRKACA (protein kinase cAMP-dependent catalytic alpha), PRKACB (protein kinase cAMP-dependent catalytic beta), PRKAR1A (protein kinase cAMP-dependent regulatory type I alpha (tissue specific extinguisher 1)), and PRKAR2A (protein kinase cAMP-dependent regulatory type II alpha); ERAL1 (Era G-protein-like 1 (E. coli)); NR2B (GRIN2B, glutamate receptor ionotropic N-methyl D-aspartate 2B); LGALS1 (lectin galactoside-binding soluble 1); TRPV1 (transient receptor potential cation channel subfamily V member 1); SCN9A (sodium channel voltage-gated type IX alpha subunit); OPRD1 (opioid receptor delta 1); OPRK1 (opioid receptor kappa 1); and OPRM1 (opioid receptor mu 1).

(iii) TRPM5/TRPM7

TRPM5 (transient receptor potential cation channel, subfamily M, member 5) and TRPM7 (transient receptor potential cation channel, subfamily M, member 7) are genes belonging to the TRPM family of transient receptor potential ion channels believed to form tetramers when functional. The relative permeability of calcium and magnesium varies widely among TRPM channels. TRPM5 is impermeable to calcium, and TRPM7 is highly permeable to both calcium and magnesium.

TRPMs are expressed on various nociceptors and respond to different sensory stimuli. TRPM5 is activated by intracellular calcium and has been associated with sensory transduction in taste cells such as umami cells. TRPM7 is reported to be involved in mechanotransduction.

(iv) TRPC1/TRPC5/TRPC6

TRPC1 (transient receptor potential cation channel subfamily C member 1), TRPC5 (transient receptor potential cation channel subfamily C member 5), and TRPC6 (transient receptor potential cation channel subfamily C member 6) are members of the TRPC family of transient receptor potential cation channels in animals. TRPC5 has been found to be involved in the action of anesthetics such as chloroform, halothane and propofol. TRPC channels in general may be activated by phospholipase C stimulation, and some channels may be activated by diacylglycerol. TRPC1 may also be activated by stretching of the membrane and TRPC5 channels may also be activated by extracellular reduced thioredoxin.

TRPC1 and TRPC6 are both reported to be involved in mechanotransduction. TRPC5 has been found to be involved in the action of anesthetics such as chloroform, halothane and propofol.

(v) TRPA1

TRPA1 (transient receptor potential cation channel subfamily A member 1) is a member of the transient receptor potential channel family. Although the specific function of this protein has not yet been determined, studies indicate the function may involve a role in signal transduction. TRPA1 is activated by a number of reactive compounds including allyl isothiocyanate, cinnamaldehyde, farnesyl thiosalicylic acid, nicotine, formalin, hydrogen peroxide, 4-hydroxynonenal, and acrolein and is considered to be a chemosensor. TRPA1 is an attractive pain target based on the finding that TRPA1 knockout mice showed near complete attenuation of formalin-induced pain behaviors. TRPA1 antagonists have demonstrated efficacy at blocking pain behaviors induced by inflammation in response to the application of complete Freund's adjuvant and formalin in animal models. Further, cold activation of TRPA1 channels has been demonstrated in vitro.

(vi) CNR1/CNR2

Cannabinoid receptor type 1 (CNR1) and type 2 (CNR2) are G protein-coupled cannabinoid receptors located in the brain that are activated by endocannabinoid neurotransmitters including anandamide and by the compound THC, found in the psychoactive drug cannabis. CNR1 receptors located on the peripheral endings of sensory neurons involved in pain transmission have been shown experimentally to attenuate the early phase or the late phase of pain behavior produced by formalin-induced chemical damage. These results further demonstrate the immunosuppressive properties of CB2 receptor agonists. CNR2 agonists may be useful for the treatment of inflammation and pain, and have been investigated particularly for forms of pain that do not respond well to conventional treatments, such as neuropathic pain.

(vii) ADRBK1

ADRBK1 (adrenergic beta receptor kinase 1), also known as GRK2, is a serine/threonine intracellular kinase that is activated by protein kinase A (PKA) and which targets beta adrenergic receptors. ADRBK1 has been associated with the modulation of inflammatory pain as the mediation of acute mu-opioid receptor desensitization in native neurons.

(viii) POMC

POMC (pro-opiomelanocortin) is a precursor polypeptide comprising 241 amino acid residues. β-endorphin, a derivative of POMC, is an endogenous opioid peptide with widespread actions in the brain, and POMC expression is a widespread measure of the response of animal models to nociceptive stimuli. POMC, as part of the melanocortin (MC) system, may play a possible direct role in nociception; melanocortin antagonists have been demonstrated to be analgesic and melanocortin agonists have been demonstrated to be hyperalgesic in animal models.

(ix) CALCA/CGRP

CALCA (calcitonin-related polypeptide alpha), also known as CGRP (calcitonin gene-related peptide), is a member of the calcitonin family of peptides. CGRP receptors are found throughout the body suggesting that the protein may modulate a variety of physiological functions in major physiological systems such as the respiratory, endocrine, gastrointestinal, immune, and cardiovascular systems. CGRP has been associated with temporomandibular joint nocioception, and CGRP levels have been shown to increase in sensory neurons during inflammation.

(x) CRF/CRH

CRF (corticotrophin releasing factor), also known as CRH (corticotrophin releasing hormone), is a 41-amino acid polypeptide that functions as a hormone and neurotransmitter involved in the stress response. Visceral nociception has been shown to upregulate CRF gene expression in various animal pain models. Activation of corticotropin releasing factor receptors is also known to be involved in stress related responses and visceral pain.

(xi) PRKACA/PRKACB/PRKAR1A/PRKAR2A

Protein kinase A (PKA) is a family of enzymes known to perform several important functions in the cell, including regulation of glycogen, sugar, and lipid metabolism. The inactive holoenzyme of PKA is a tetramer composed of two regulatory units encoded by PRKAR1A (protein kinase cAMP-dependent regulatory type I alpha) and PRKAR2A (protein kinase cAMP-dependent regulatory type II alpha) and two catalytic subunits encoded by PRKACA (protein kinase cAMP-dependent catalytic alpha) and PRKACB (protein kinase cAMP-dependent catalytic beta). The activation of PKA by cAMP causes the dissociation of the inactive holoenzyme into a dimer of regulatory subunits bound to four cAMP and two free monomeric catalytic subunits. Activated PKA may modify the activity of different target proteins through direct phosphorylation of the target proteins.

PKA has been associated with the regulation of CNS nociceptive neurons following peripheral painful stimuli in animal models. Intrathecal injection of protein kinase A inhibitor has been shown to reverse mechanical hyperalgesia. PKA cascades have been associated with the regulation of phospho-CREB, a protein associated with hyperalgia and neuropathic pain mechanisms.

(xii) ERAL 1

ERAL1 (Era G-protein-like 1) is a putative GTPase with possible roles in cell cycle control. ERAL1 was found to be upregulated in reaction to spinal cord damage in experimental nerve regeneration models, and may play a role as a signaling factor in nocioception as well.

(xiii) NR2B/GRIN2B

NR2B (N-methyl D-aspartate receptor subtype 2B), also known as GRIN2B (glutamate receptor, ionotropic, N-methyl D-aspartate (NMDA) 2B), is a type of N-methyl-D-aspartate (NMDA) receptor. NMDA receptors are a class of ionotropic glutamate receptors that are involved in long-term potentiation, an activity-dependent increase in the efficiency of synaptic transmission thought to underlie certain kinds of memory and learning. NR2B has been associated with the modulation of thermal hyperalgia in animal models.

(xiv) LGALS1

LGALS1 (lectin galactoside-binding soluble 1), also known as galectin-1, is a protein from the galectin group. The galectins are a family of beta-galactoside-binding proteins implicated in modulating cell-cell and cell-matrix interactions. Galectin-1 is expressed extensively in peripheral projecting neurons, and is associated with the potentiation of neuropathic pain in the dorsal horn. Mice lacking galectin-1 were shown to have reduced thermal sensitivity.

(xv) TRPV1

TRPV1 (transient receptor potential cation channel subfamily V member 1), also known as capsaicin receptor, is a member of the TRPV group of transient receptor potential family of ion channels. TRPV1 is a nonselective cation channel that may be activated by a wide variety of exogenous and endogenous physical and chemical stimuli. The best-known activators of TRPV1 are heat greater than 43° C. and capsaicin, the pungent compound in hot chili peppers. Activation of TRPV1 results in a painful, burning sensation. TRPV1 receptors are found mainly in the nociceptive neurons of the peripheral nervous system, but they have also been described in many other tissues, including the central nervous system. TRPV1 is involved in the transmission and modulation of pain (nociception), as well as the integration of diverse painful stimuli.

(xvi) SCN9A

SCN9A (sodium channel voltage-gated type IX alpha subunit), also known as Naᵥ1.7 is a sodium ion channel that is expressed at high levels in nociceptive dorsal root ganglion (DRG) neurons. SCN9A amplifies generator potentials produced by the stimulation of nociceptors nerve endings, and function as a major sodium channel in peripheral nociception.

Knockout mice lacking SCN9A in their nociceptors showed reduced response to inflammatory pain, yet remained responsive to neuropathic pain, indicating that SCN9A plays an important role in setting the inflammatory pain threshold. SCN9A mutations in multiple families are associated with erythromelagia, an inherited disorder characterized by symmetrical burning pain of the feet, lower legs, and hands. Loss of SCN9A function due to missense mutations has also been implicated in the congenital inability to sense pain.

(xvii) OPRD1/OPRK1/OPRM1

OPRD1 (opioid receptor delta 1), OPRK1 (opioid receptor kappa 1), and OPRM1 (opioid receptor mu 1) are opioid receptors belonging to a group of G protein-coupled receptors with opioids as ligands. Endogenous opioids which activate the opioid receptors include dynorphins, enkephalins, endorphins, endomorphins and nociceptin.

OPRM1 is a μ-opioid receptor (MOR) with a high affinity for enkephalins and beta-endorphin but low affinity for dynorphins. The prototypical μ opioid receptor agonist is the opium alkaloid morphine. Activation of the p receptor by an agonist such as morphine or endogenous opioids results in supraspinal analgesia.

OPRD1 is a δ-opioid receptor (DOR) that includes enkephalins as endogenous ligands. Activation of OPRD1 produces some analgesia, although less than the analgesia resulting from the activation of OPRM1 mu-opioid agonists.

OPRK1 is a κ-opioid receptor (KOR) which binds the opioid peptide dynorphin as its primary endogenous ligand. OPRK1 is widely distributed in the brain (hypothalamus, periaqueductal gray, and claustrum), spinal cord (substantia gelatinosa), and in pain neurons. OPRK1 activation produces an analgesic effect as well as associated side effects such as sedation and dysphoria.

Opioid receptors are associated with the modulation of a wide range of nociception responses. Each receptor presents a distinct pattern of activities, with OPRM1 influencing responses to mechanical, chemical and thermal nociception at a supraspinal level, OPRK1 involved in spinally mediated thermal nociception and chemical visceral pain, and OPRD1 modulating mechanical nociception and inflammatory pain.

The identity of the sensory-related protein in which a chromosomal sequence is edited can and will vary. In general, the exemplary sensory-related protein in which a chromosomal sequence is edited may be TRPM5, TRPM7, TRPC1, TRPC5, TRPC6, TRPA1, CNR1, CNR2, POMC, CALCA, CRF, PRKACA, PRKACB, PRKAR1A, PRKAR2A, ERAL1, NR2B, LGALS1, TRPV1, SCN9A, OPRM1, OPRD1, OPRK1, and any combination thereof.

In one aspect, the chromosomal sequences of any combination of any two sensory-related proteins may be edited using a zinc finger nuclease-mediated process. In other aspects, the chromosomal sequences of any combination of any three exemplary sensory-related proteins, any four exemplary sensory-related proteins, any five exemplary sensory-related proteins, any six exemplary sensory-related proteins, any seven exemplary sensory-related proteins, any eight exemplary sensory-related proteins, any nine exemplary sensory-related proteins, any ten exemplary sensory-related proteins, any eleven exemplary sensory-related proteins, any twelve exemplary sensory-related proteins, any thirteen exemplary sensory-related proteins, any fourteen exemplary sensory-related proteins, any fifteen exemplary sensory-related proteins, any sixteen exemplary sensory-related proteins, any seventeen exemplary sensory-related proteins, any eighteen exemplary sensory-related proteins, any nineteen exemplary sensory-related proteins, any twenty exemplary sensory-related proteins, any twenty-one exemplary sensory-related proteins, or any twenty-two exemplary sensory-related proteins may be edited using a zinc finger nuclease-mediated process. In yet another aspect, the chromosomal sequences of any combination of all twenty-two exemplary sensory-related proteins may be edited using a zinc finger nuclease-mediated process.

Exemplary genetically modified animals may comprise one, two, three, four, five, six, seven, eight, nine, ten, eleven, twelve, thirteen, fourteen, fifteen, sixteen, seventeen, eighteen, nineteen, twenty, twenty-one, twenty-two or twenty-three inactivated chromosomal sequences encoding a sensory-related protein and zero, one, two, three, four, five, six, seven, eight, nine, ten, eleven, twelve, thirteen, fourteen, fifteen, sixteen, seventeen, eighteen, nineteen, twenty, twenty-one, twenty-two or twenty-three chromosomally integrated sequences encoding orthologous or modified sensory-related proteins.

### (b) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In another iteration of the invention, the animal does not comprise a genetically modified mouse. In each of the foregoing iterations of suitable animals for the invention, the animal does not include exogenously introduced, randomly integrated transposon sequences.

### (c) Sensory-Related Protein

The sensory-related protein may be from any of the animals listed above. Furthermore, the sensory-related protein may be a human sensory-related protein. Additionally, the sensory-related protein may be a bacterial, fungal, or plant sensory-related protein. The type of animal and the source of the protein can and will vary. The protein may be endogenous or exogenous (such as an orthologous protein). As an example, the genetically modified animal may be a rat, cat, dog, or pig, and the orthologous sensory-related protein may be human. Alternatively, the genetically modified animal may be a rat, cat, or pig, and the orthologous sensory-related protein may be canine. One of skill in the art will readily appreciate that numerous combinations are possible.

Additionally, the sensory-related gene may be modified to include a tag or reporter gene as are well-known. Reporter genes include those encoding selectable markers such as cloramphenicol acetyltransferase (CAT) and neomycin phosphotransferase (neo), and those encoding a fluorescent protein such as green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalamal), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the targeted gene to create a gene fusion. A reporter sequence can be integrated in a targeted manner in the targeted gene, for example the reporter sequences may be integrated specifically at the 5′ or 3′ end of the targeted gene. The two genes are thus under the control of the same promoter elements and are transcribed into a single messenger RNA molecule. Alternatively, the reporter gene may be used to monitor the activity of a promoter in a genetic construct, for example by placing the reporter sequence downstream of the target promoter such that expression of the reporter gene is under the control of the target promoter, and activity of the reporter gene can be directly and quantitatively measured, typically in comparison to activity observed under a strong consensus promoter. It will be understood that doing so may or may not lead to destruction of the targeted gene.

### II. Genetically Modified Cells

A further aspect of the present disclosure provides genetically modified cells or cell lines comprising at least one edited chromosomal sequence encoding a sensory-related protein. The genetically modified cell or cell line may be derived from any of the genetically modified animals disclosed herein. Alternatively, the chromosomal sequence coding a sensory-related protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CVI-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TRI cells. For an extensive list of mammalian cell lines, those of ordinary skill in the art may refer to the American Type Culture Collection catalog (ATCC®, Mamassas, Va).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### (III) Zinc Finger-Mediated Genome Editing

In general, the genetically modified animal or cell detailed above in sections (I) and (II), respectively, is generated using a zinc finger nuclease-mediated genome editing process. The process for editing a chromosomal sequence comprises: (a) introducing into an embryo or cell at least one nucleic acid encoding a zinc finger nuclease that recognizes a target sequence in the chromosomal sequence and is able to cleave a site in the chromosomal sequence, and, optionally, (i) at least one donor polynucleotide comprising a sequence for integration flanked by an upstream sequence and a downstream sequence that share substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the embryo or cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence.

Components of the zinc finger nuclease-mediated method are described in more detail below.

### (a) Zinc Finger Nuclease

The method comprises, in part, introducing into an embryo or cell at least one nucleic acid encoding a zinc finger nuclease. Typically, a zinc finger nuclease comprises a DNA binding domain (i.e., zinc finger) and a cleavage domain (i.e., nuclease). The DNA binding and cleavage domains are described below. The nucleic acid encoding a zinc finger nuclease may comprise DNA or RNA. For example, the nucleic acid encoding a zinc finger nuclease may comprise mRNA. When the nucleic acid encoding a zinc finger nuclease comprises mRNA, the mRNA molecule may be 5′ capped. Similarly, when the nucleic acid encoding a zinc finger nuclease comprises mRNA, the mRNA molecule may be polyadenylated. An exemplary nucleic acid according to the method is a capped and polyadenylated mRNA molecule encoding a zinc finger nuclease. Methods for capping and polyadenylating mRNA are known in the art.

(i) Zinc Finger Binding Domain

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain may have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm of described in U.S. Pat. No. 6,453,242 may be used to design a zinc finger binding domain to target a preselected sequence. Alternative methods, such as rational design using a nondegenerate recognition code table may also be used to design a zinc finger binding domain to target a specific sequence (Sera et al. (2002) Biochemistry 41:7074-7081). Publically available web-based tools for identifying potential target sites in DNA sequences and designing zinc finger binding domains may be found at http://www.zincfingertools.org and http://bindr.gdcb.iastate.edu/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize a DNA sequence ranging from about 3 nucleotides to about 21 nucleotides in length, or from about 8 to about 19 nucleotides in length. In general, the zinc finger binding domains of the zinc finger nucleases disclosed herein comprise at least three zinc finger recognition regions (i.e., zinc fingers). In one embodiment, the zinc finger binding domain may comprise four zinc finger recognition regions. In another embodiment, the zinc finger binding domain may comprise five zinc finger recognition regions. In still another embodiment, the zinc finger binding domain may comprise six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers of the protein.

In some embodiments, the zinc finger nuclease may further comprise a nuclear localization signal or sequence (NLS). A NLS is an amino acid sequence which facilitates targeting the zinc finger nuclease protein into the nucleus to introduce a double stranded break at the target sequence in the chromosome. Nuclear localization signals are known in the art. See, for example, Makkerh et al. (1996) Current Biology 6:1025-1027.

(ii) Cleavage Domain

A zinc finger nuclease also includes a cleavage domain. The cleavage domain portion of the zinc finger nucleases disclosed herein may be obtained from any endonuclease or exonuclease. Non-limiting examples of endonucleases from which a cleavage domain may be derived include, but are not limited to, restriction endonucleases and homing endonucleases. See, for example, 2002-2003 Catalog, New England Biolabs, Beverly, Mass.; and Belfort et al. (1997) Nucleic Acids Res. 25:3379-3388 or www.neb.com. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease may comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs may intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease may comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is Fok I. This particular enzyme is active as a dimmer (Bitinaite et al. (1998) Proc. Natl. Acad. Sci. USA 95: 10, 570-10, 575). Accordingly, for the purposes of the present disclosure, the portion of the Fok I enzyme used in a zinc finger nuclease is considered a cleavage monomer. Thus, for targeted double-stranded cleavage using a Fok I cleavage domain, two zinc finger nucleases, each comprising a Fokl cleavage monomer, may be used to reconstitute an active enzyme dimer. Alternatively, a single polypeptide molecule containing a zinc finger binding domain and two Fok I cleavage monomers may also be used.

In certain embodiments, the cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains. Exemplary engineered cleavage monomers of Fok I that form obligate heterodimers include a pair in which a first cleavage monomer includes mutations at amino acid residue positions 490 and 538 of Fok I and a second cleavage monomer that includes mutations at amino-acid residue positions 486 and 499.

Thus, in one embodiment, a mutation at amino acid position 490 replaces Glu (E) with Lys (K); a mutation at amino acid residue 538 replaces Iso (I) with Lys (K); a mutation at amino acid residue 486 replaces Gln (Q) with Glu (E); and a mutation at position 499 replaces Iso (I) with Lys (K). Specifically, the engineered cleavage monomers may be prepared by mutating positions 490 from E to K and 538 from I to K in one cleavage monomer to produce an engineered cleavage monomer designated “E490K:1538K” and by mutating positions 486 from Q to E and 499 from Ito L in another cleavage monomer to produce an engineered cleavage monomer designated “Q486E:I499L.” The above described engineered cleavage monomers are obligate heterodimer mutants in which aberrant cleavage is minimized or abolished. Engineered cleavage monomers may be prepared using a suitable method, for example, by site-directed mutagenesis of wild-type cleavage monomers (Fok I) as described in U.S. Patent Publication No. 20050064474 (see Example 5).

The zinc finger nuclease described above may be engineered to introduce a double stranded break at the targeted site of integration. The double stranded break may be at the targeted site of integration, or it may be up to 1, 2, 3, 4, 5, 10, 15, 20, 25, 30, 35, 40, 45, 50, 100, or 1000 nucleotides away from the site of integration. In some embodiments, the double stranded break may be up to 1, 2, 3, 4, 5, 10, 15, or 20 nucleotides away from the site of integration. In other embodiments, the double stranded break may be up to 10, 15, 20, 25, 30, 35, 40, 45, or 50 nucleotides away from the site of integration. In yet other embodiments, the double stranded break may be up to 50, 100, or 1000 nucleotides away from the site of integration.

### (b) Optional Donor Polynucleotide

The method for editing chromosomal sequences encoding sensory-related proteins may further comprise introducing at least one donor polynucleotide comprising a sequence encoding a sensory-related protein into the embryo or cell. A donor polynucleotide comprises at least three components: the sequence coding the sensory-related protein, an upstream sequence, and a downstream sequence. The sequence encoding the protein is flanked by the upstream and downstream sequence, wherein the upstream and downstream sequences share sequence similarity with either side of the site of integration in the chromosome.

Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary donor polynucleotide comprising the sequence encoding a sensory-related protein may be a BAC.

The sequence of the donor polynucleotide that encodes the sensory-related protein may include coding (i.e., exon) sequence, as well as intron sequences and upstream regulatory sequences (such as, e.g., a promoter). Depending upon the identity and the source of the sensory-related protein, the size of the sequence encoding the sensory-related protein can and will vary. For example, the sequence encoding the sensory-related protein may range in size from about 1 kb to about 5,000 kb.

The donor polynucleotide also comprises upstream and downstream sequence flanking the sequence encoding the sensory-related protein. The upstream and downstream sequences in the donor polynucleotide are selected to promote recombination between the chromosomal sequence of interest and the donor polynucleotide. The upstream sequence, as used herein, refers to a nucleic acid sequence that shares sequence similarity with the chromosomal sequence upstream of the targeted site of integration. Similarly, the downstream sequence refers to a nucleic acid sequence that shares sequence similarity with the chromosomal sequence downstream of the targeted site of integration. The upstream and downstream sequences in the donor polynucleotide may share about 75%, 80%, 85%, 90%, 95%, or 100% sequence identity with the targeted chromosomal sequence. In other embodiments, the upstream and downstream sequences in the donor polynucleotide may share about 95%, 96%, 97%, 98%, 99%, or 100% sequence identity with the targeted chromosomal sequence. In an exemplary embodiment, the upstream and downstream sequences in the donor polynucleotide may share about 99% or 100% sequence identity with the targeted chromosomal sequence.

An upstream or downstream sequence may comprise from about 50 by to about 2500 bp. In one embodiment, an upstream or downstream sequence may comprise about 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. An exemplary upstream or downstream sequence may comprise about 200 by to about 2000 bp, about 600 by to about 1000 bp, or more particularly about 700 by to about 1000 bp.

In some embodiments, the donor polynucleotide may further comprise a marker. Such a marker may make it easy to screen for targeted integrations. Non-limiting examples of suitable markers include restriction sites, fluorescent proteins, or selectable markers.

One of skill in the art would be able to construct a donor polynucleotide as described herein using well-known standard recombinant techniques (see, for example, Sambrook et al., 2001 and Ausubel et al., 1996).

In the method detailed above for integrating a sequence encoding the sensory-related protein, a double stranded break introduced into the chromosomal sequence by the zinc finger nuclease is repaired, via homologous recombination with the donor polynucleotide, such that the sequence encoding the sensory-related protein is integrated into the chromosome. The presence of a double-stranded break facilitates integration of the sequence into the chromosome. A donor polynucleotide may be physically integrated or, alternatively, the donor polynucleotide may be used as a template for repair of the break, resulting in the introduction of the sequence encoding the sensory-related protein as well as all or part of the upstream and downstream sequences of the donor polynucleotide into the chromosome. Thus, endogenous chromosomal sequence may be converted to the sequence of the donor polynucleotide.

### (c) Optional Exchange Polynucleotide

The method for editing chromosomal sequences encoding sensory-related protein may further comprise introducing into the embryo or cell at least one exchange polynucleotide comprising a sequence that is substantially identical to the chromosomal sequence at the site of cleavage and which further comprises at least one specific nucleotide change.

Typically, the exchange polynucleotide will be DNA. The exchange polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exchange polynucleotide may be a DNA plasmid.

The sequence in the exchange polynucleotide is substantially identical to a portion of the chromosomal sequence at the site of cleavage. In general, the sequence of the exchange polynucleotide will share enough sequence identity with the chromosomal sequence such that the two sequences may be exchanged by homologous recombination. For example, the sequence in the exchange polynucleotide may have at least about 80, 81, 82, 83, 84, 85, 86, 87, 88, 89, 90, 91, 92, 93, 94, 95, 96, 97, 98, or 99% sequence identity with a portion of the chromosomal sequence.

Importantly, the sequence in the exchange polynucleotide comprises at least one specific nucleotide change with respect to the sequence of the corresponding chromosomal sequence. For example, one nucleotide in a specific codon may be changed to another nucleotide such that the codon codes for a different amino acid. In one embodiment, the sequence in the exchange polynucleotide may comprise one specific nucleotide change such that the encoded protein comprises one amino acid change. In other embodiments, the sequence in the exchange polynucleotide may comprise two, three, four, or more specific nucleotide changes such that the encoded protein comprises one, two, three, four, or more amino acid changes. In still other embodiments, the sequence in the exchange polynucleotide may comprise a three nucleotide deletion or insertion such that the reading frame of the coding reading is not altered (and a functional protein is produced). The expressed protein, however, would comprise a single amino acid deletion or insertion.

The length of the sequence in the exchange polynucleotide that is substantially identical to a portion of the chromosomal sequence at the site of cleavage can and will vary. In general, the sequence in the exchange polynucleotide may range from about 50 by to about 10,000 by in length. In various embodiments, the sequence in the exchange polynucleotide may be about 100, 200, 400, 600, 800, 1000, 1200, 1400, 1600, 1800, 2000, 2200, 2400, 2600, 2800, 3000, 3200, 3400, 3600, 3800, 4000, 4200, 4400, 4600, 4800, or 5000 by in length. In other embodiments, the sequence in the exchange polynucleotide may be about 5500, 6000, 6500, 6000, 6500, 7000, 7500, 8000, 8500, 9000, 9500, or 10,000 by in length.

One of skill in the art would be able to construct an exchange polynucleotide as described herein using well-known standard recombinant techniques (see, for example, Sambrook et al., 2001 and Ausubel et al., 1996).

In the method detailed above for modifying a chromosomal sequence, a double stranded break introduced into the chromosomal sequence by the zinc finger nuclease is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the endogenous chromosomal sequence may be converted to the sequence of the exchange polynucleotide. The changed nucleotide(s) may be at or near the site of cleavage. Alternatively, the changed nucleotide(s) may be anywhere in the exchanged sequences. As a consequence of the exchange, however, the chromosomal sequence is modified.

### (d) Delivery of Nucleic Acids

To mediate zinc finger nuclease genomic editing, at least one nucleic acid molecule encoding a zinc finger nuclease and, optionally, at least one exchange polynucleotide or at least one donor polynucleotide are delivered to the embryo or the cell of interest. Typically, the embryo is a fertilized one-cell stage embryo of the species of interest.

Suitable methods of introducing the nucleic acids to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the nucleic acids may be introduced into an embryo by microinjection. The nucleic acids may be microinjected into the nucleus or the cytoplasm of the embryo. In another embodiment, the nucleic acids may be introduced into a cell by nucleofection.

In embodiments in which both a nucleic acid encoding a zinc finger nuclease and a donor (or exchange) polynucleotide are introduced into an embryo or cell, the ratio of donor (or exchange) polynucleotide to nucleic acid encoding a zinc finger nuclease may range from about 1:10 to about 10:1. In various embodiments, the ratio of donor (or exchange) polynucleotide to nucleic acid encoding a zinc finger nuclease may be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. In one embodiment, the ratio may be about 1:1.

In embodiments in which more than one nucleic acid encoding a zinc finger nuclease and, optionally, more than one donor (or exchange) polynucleotide are introduced into an embryo or cell, the nucleic acids may be introduced simultaneously or sequentially. For example, nucleic acids encoding the zinc finger nucleases, each specific for a distinct recognition sequence, as well as the optional donor (or exchange) polynucleotides, may be introduced at the same time. Alternatively, each nucleic acid encoding a zinc finger nuclease, as well as the optional donor (or exchange) polynucleotides, may be introduced sequentially

### (e) Culturing the Embryo or Cell

The method of inducing genomic editing with a zinc finger nuclease further comprises culturing the embryo or cell comprising the introduced nucleic acid(s) to allow expression of the zinc finger nuclease. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise the edited chromosomal sequence encoding the sensory-related protein in every cell of the body.

Similarly, cells comprising the introduced nucleic acids may be cultured using standard procedures to allow expression of the zinc finger nuclease. Standard cell culture techniques are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

Upon expression of the zinc finger nuclease, the chromosomal sequence may be edited. In cases in which the embryo or cell comprises an expressed zinc finger nuclease but no donor (or exchange) polynucleotide, the zinc finger nuclease recognizes, binds, and cleaves the target sequence in the chromosomal sequence of interest. The double-stranded break introduced by the zinc finger nuclease is repaired by an error-prone non-homologous end-joining DNA repair process. Consequently, a deletion, insertion, or nonsense mutation may be introduced in the chromosomal sequence such that the sequence is inactivated.

In cases in which the embryo or cell comprises an expressed zinc finger nuclease as well as a donor (or exchange) polynucleotide, the zinc finger nuclease recognizes, binds, and cleaves the target sequence in the chromosome. The double-stranded break introduced by the zinc finger nuclease is repaired, via homologous recombination with the donor (or exchange) polynucleotide, such that the sequence in the donor polynucleotide is integrated into the chromosomal sequence (or a portion of the chromosomal sequence is converted to the sequence in the exchange polynucleotide). As a consequence, a sequence may be integrated into the chromosomal sequence (or a portion of the chromosomal sequence may be modified).

The genetically modified animals disclosed herein may be crossbred to create animals comprising more than one edited chromosomal sequence or to create animals that are homozygous for one or more edited chromosomal sequences. For example, two animals comprising the same edited chromosomal sequence may be crossbred to create an animal homozygous for the edited chromosomal sequence. Alternatively, animals with different edited chromosomal sequences may be crossbred to create an animal comprising both edited chromosomal sequences.

For example, animal A comprising an inactivated trpm5 chromosomal sequence may be crossed with animal B comprising a chromosomally integrated sequence encoding a human TRPM5 protein to give rise to a “humanized” TRPM5 offspring comprising both the inactivated trpm5 chromosomal sequence and the chromosomally integrated human TRPM5 sequence. Similarly, an animal comprising an inactivated trpm5 cnr1 chromosomal sequence may be crossed with an animal comprising a chromosomally integrated sequence encoding the human sensory-related CNR1 protein to generate “humanized” sensory-related CNR1 offspring. Moreover, a humanized FMR1 animal may be crossed with a humanized CNR1 animal to create a humanized FMR1/CNR1 offspring. Those of skill in the art will appreciate that many combinations are possible. Exemplary combinations of chromosomal sequences are presented above.

In other embodiments, an animal comprising an edited chromosomal sequence disclosed herein may be crossbred to combine the edited chromosomal sequence with other genetic backgrounds. By way of non-limiting example, other genetic backgrounds may include wild-type genetic backgrounds, genetic backgrounds with deletion mutations, genetic backgrounds with another targeted integration, and genetic backgrounds with non-targeted integrations. Suitable integrations may include without limit nucleic acids encoding drug transporter proteins, Mdr protein, and the like.

### (IV) Applications

A further aspect of the present disclosure encompasses a method for assessing an effect of an agent such as a pharmaceutically active ingredient, a drug, a toxin, or a chemical. Suitable agents include without limit pharmaceutically active ingredients, drugs, foods, food additives, pesticides, herbicides, toxins, industrial chemicals, household chemicals, and other environmental chemicals. For example, the effect of an agent may be measured in a “humanized” genetically modified animal, such that the information gained therefrom may be used to predict the effect of the agent in a human. In general, the method comprises administering the agent to a genetically modified animal comprising at least one inactivated chromosomal sequence encoding a sensory-related protein and at least one chromosomally integrated sequence encoding an orthologous sensory-related protein with the agent, and comparing a selected parameter obtained from the genetically modified animal to the selected parameter obtained from a wild-type animal administered the same agent.

Non-limiting examples of selected parameters include: (a) rate of elimination of the agent or at least one agent metabolite; (b) circulatory levels of the agent or at least one agent metabolite; (c) bioavailability of the agent or at least one agent metabolite; (d) rate of metabolism of the agent or at least one agent metabolite; (e) rate of clearance of the agent or at least one agent metabolite; (f) toxicity of the agent or at least one agent metabolite; (g) efficacy of the agent or at least one agent metabolite; (h) disposition of the agent or at least one agent metabolite; (i) extrahepatic contribution to metabolic rate and clearance of the agent or at least one agent metabolite; and (j) ability of the agent to modify an incidence or indication of a sensory disorder in the genetically modified animal. Non-limiting examples of a sensory disorder include a nociception disorder, a taste disorder, or any combination thereof.

The agent may be a therapeutic treatment for a sensory disorder, including but not limited to administering of one or more novel candidate therapeutic compounds, administering a novel combination of established therapeutic compounds, a novel therapeutic method, and any combination thereof. Non-limiting examples of novel therapeutic methods include various drug delivery mechanisms such as oral or injected therapeutic compositions, drug-releasing implants, nanotechnology applications in drug therapy, surgery, and combinations thereof.

For example, an ADME-Tox profile of an agent may be assessed using a genetically modified animal. The ADME-Tox profile may include assessments of at least one or more physiologic and metabolic consequences of administering the agent. In addition, the ADME-Tox profile may assess behavioral effects such as addiction or depression in response to the agent.

A further aspect of the present disclosure encompasses a method for assessing an indication of a sensory disorder in an animal model comprising a genetically modified animal comprising at least one edited chromosomal sequence encoding a sensory-related protein. This method includes comparing a selected parameter obtained from the animal model to the selected parameter obtained from a wild-type animal. Non-limiting examples of the selected parameter used for assessing at least one indication of a sensory disorder include a) spontaneous behaviors; b) performance during behavioral testing; c) physiological anomalies; d) abnormalities in tissues or cells; e) biochemical function; f) molecular structures; and combinations thereof.

The sensory disorders assessed by the method may include any one or more of the nociception disorders and taste disorders described above. Non-limiting examples of nociception disorders include allodynia; neuralgia; HSAN-1 such as hereditary sensory radicular neuropathy, ulcero-mutilating neuropathy, thevenard syndrome, familial trophoneurosis, mal perforant du pied, familial syringomyelia, and Charcot-Marie-Tooth type 2B syndrome; HSAN-2 such as congenital sensory neuropathy or Morvan's disease; HSAN-3 such as familial dysautonomia (FD) or Riley-Day syndrome; HSAN-4 such as congenital insensitivity to pain with anhidrosis (CIPA); and HSAN-5 such as congenital insensitivity to pain with partial anhidrosis. Non-limiting examples of taste disorders include dysgeusia, hypogeusia, and ageusia.

The at least one indication of the sensory disorder may occur spontaneously in the animal model, or may be promoted by exposure to an exogenous agent including but not limited to a nociception stimulus, a taste stimulus, a sensory-related protein, a sensory-related agonist, and a sensory-related antagonist.

Nociception stimuli may include any protocols known in the art to induce a behavioral or biochemical indication of nociception, including but not limited to mechanical nociception stimuli, surgical procedures, the injection or topical application of noxious chemical stimuli, thermal nociception stimuli, electrical nociception stimuli, and stress stimuli. Non-limiting examples of mechanical nociception stimuli include sciatic nerve chronic constriction injury, spinal cord injury, paw pressure, repeated trauma, mechanical stimulation (pinching), paw bending, von Frey test, tail-clip test, CO₂ pulses to a nasal cavity, colorectal distention, and compression of dorsal root ganglia. Non-limiting examples of surgical procedures include pulp exposures in the maxillary and mandibular first molars or other teeth, spinal nerve transactions, exposure of a dorsal root ganglion, loose ligation of a dorsal root ganglion, and chronic gut suture exposure. Non-limiting examples of noxious chemical stimuli include formalin, acetic acid, zymosan, hypotonic saline, kainate, capsaicin, triptan, Freund's adjuvant, mustard oil, bee venom, carrageenan, collagen II, paclitaxel, vincristine, and hydrochloric acid. Non-limiting examples of thermal nociception stimuli include hot plate testing, heat irradiation stimulation, and noxious heat stimulation of an exposed skin nerve. Non-limiting examples of electrical nociception stimuli include foot shocking, application of radiofrequency current, and electroacupuncture. Non-limiting examples of stress stimuli include forced swimming, cold swimming, platform shaker stimuli, loud noises, and immobilization stress.

Taste stimuli may include any protocols known in the art to induce a behavioral or biochemical indication of taste sensation, including but not limited to conditioned taste aversion (CTA); novel gustatory stimuli; and exposure to various flavored compounds. Non-limiting examples of flavored compounds include bitter-tasting compounds such as denatonium and quinine; sweet-tasting compounds such as sucrose and other polysaccharide compounds; salty-tasting compounds such as sodium chloride; sour-tasting compounds such as lemon juice; savory or umami-tasting compounds such as monosodium glutamate (MSG) and other glutamate compounds; ethanol; and cinnamon.

Suitable sensory-related proteins may include any one or more of sensory-related proteins described above, including but not limited to TRPM5, TRPM7, TRPC1, TRPC5, TRPC6, TRPA1, CNR1, CNR2, POMC, CALCA, CRF, PRKACA, PRKACB, PRKAR1A, PRKAR2A, ERAL1, NR2B, LGALS1, TRPV1, SCN9A, OPRM1, OPRD1, OPRK1, and any combination thereof. The sensory-related agonist may be any compound that interacts with a sensory-related receptor and triggers a cellular response by the cell such as the enhanced production or release of a sensory-related protein by the cell, the modification of neuronal signaling, and combinations thereof. The sensory-related antagonist may be any compound that inhibits the response of a cell having sensory-related receptors. The sensory-related antagonist may be a compound that binds to the sensory-related receptor without triggering a cellular response, a compound that interferes with the binding of an endogenous sensory-related agonist by binding to the sensory-related agonist, by inhibiting the production of the endogenous sensory-related agonist, by altering the structure of the endogenous sensory-related agonist, or by otherwise disrupting the binding of the endogenous sensory-related agonist to the sensory-related receptor.

Spontaneous behavior may be assessed using any one or more methods of spontaneous behavioral observation known in the art. In general, any spontaneous behavior within a known behavioral repertoire of an animal may be observed, including movement, posture, social interaction, rearing, sleeping, blinking, eating, drinking, urinating, defecating, mating, and aggression. An extensive battery of observations for quantifying the spontaneous behavior of mice and rats is well-known in the art, including but not limited to home-cage observations such as body position, respiration, tonic involuntary movement, unusual motor behavior such as pacing or rocking, catatonic behavior, vocalization, palpebral closure, mating frequency, running wheel behavior, nest building, and frequency of aggressive interactions.

Performance during behavioral testing may be assessed using any number of behavioral tests known in the art. The particular type of performance test may depend upon at least one of several factors including the behavioral repertoire of the animal and the purpose of the testing. Non-limiting examples of tests for assessing the reflex function of rats include assessments of approach response, touch response, eyelid reflex, pinna reflex, sound response, tail pinch response, pupillary reflex, and righting reflex. Non-limiting examples of behavioral tests suitable for assessing the motor function of rats includes open field locomotor activity assessment, the rotarod test, the grip strength test, the cylinder test, the limb-placement or grid walk test, the vertical pole test, the Inverted grid test, the adhesive removal test, the painted paw or catwalk (gait) tests, the beam traversal test, and the inclined plane test. Non-limiting examples of behavioral tests suitable for assessing the long-term memory function of rats include the elevated plus maze test, the Morris water maze swim test, contextual fear conditioning, the Y-maze test, the T-maze test, the novel object recognition test, the active avoidance test, the passive (inhibitory) avoidance test, the radial arm maze test, the two-choice swim test, the hole board test, the olfactory discrimination (go-no-go) test, and the pre-pulse inhibition test. Non-limiting examples of behavioral tests suitable for assessing the anxiety of rats include the open field locomotion assessment, observations of marble-burying behavior, the elevated plus maze test, the light/dark box test. Non-limiting examples of behavioral tests suitable for assessing the depression of rats includes the forced swim test, the tail suspension test, the hot plate test, the tail suspension test, anhedonia observations, and the novelty suppressed feeding test.

Physiological anomalies may include any difference in physiological function between a genetically modified animal and a wild-type animal. Non-limiting examples of physiological functions include homeostasis, metabolism, sensory function, neurological function, musculoskeletal function, cardiovascular function, respiratory function, dermatological function, renal function, reproductive functions, immunological function, and endocrinological function. Numerous measures of physiological function are well-known in the art.

Abnormalities in tissues or cells may include any difference in the structure or function of a tissue or cell of a genetically modified animal and the corresponding structure or function of a wild-type animal. Non-limiting examples of cell or tissue abnormalities include cell hypertrophy, tissue hyperplasia, neoplasia, hypoplasia, aplasia, hypotrophy, dysplasia, overproduction or underproduction of cell products, abnormal neuronal discharge frequency, and changes in synaptic density of neurons.

Non-limiting examples of biochemical functions may include enzyme function, cell signaling function, maintenance of homeostasis, cellular respiration; methods of assessing biochemical functions are well known in the art. Molecular structures may be assessed using any method known in the art including microscopy such as dual-photon microscopy and scanning electron microscopy, and immunohistological techniques such as Western blot and ELISA.

A additional aspect provides a method for assessing a side effect of a therapeutic compound comprising administering the therapeutic compound to an animal model and assessing at least one or more behaviors chosen from learning, memory, anxiety, depression, addiction, sensory-motor function, taste preference, and odor preference. The animal model may be chosen from a genetically modified animal and a wild-type animal. The genetically modified animal comprises at least one edited chromosomal sequence encoding a sensory-related protein. The therapeutic compound is chosen from a novel therapeutic compound and a novel combination of known therapeutic agents. Any of the methods described above to measure spontaneous behavior or performance during behavioral tests may be used to assess the side effect.

In this method, the therapeutic compound may be self-administered, or the therapeutic compound may be administered by another. The animal model may be contacted with the therapeutic compound using administration methods including oral ingestion, epidermal absorption, injection, absorption through the mucous membranes of the oral cavity, rectum, nasal cavity, lungs, or vagina, and any other suitable administration method known in the art. If the therapeutic compound is administered using oral ingestion, the therapeutic compound may be incorporated in an amount of water, food, or supplemental material such as a chewable or lickable object and provided to the animal model.

Also provided are methods to assess an effect of an agent in an isolated cell comprising at least one edited chromosomal sequence encoding a sensory-related protein, as well as methods of using lysates of such cells (or cells derived from a genetically modified animal disclosed herein) to assess the effect of an agent. For example, the role of a particular sensory-related protein in the metabolism of a particular agent may be determined using such methods. Similarly, substrate specificity and pharmacokinetic parameter may be readily determined using such methods. Those of skill in the art are familiar with suitable tests and/or procedures.

Yet another aspect encompasses a method for assessing the therapeutic efficacy of a potential gene therapy strategy. That is, a chromosomal sequence encoding a sensory-related protein may be modified such that the susceptibility to a sensory disorder or the indications of the disorder are reduced or eliminated. In particular, the method comprises editing a chromosomal sequence encoding a sensory-related protein such that an altered protein product is produced. The genetically modified animal may be exposed to an exogenous agent including but not limited to a nociception stimulus, a taste stimulus, a sensory-related protein, a sensory-related agonist, and a sensory-related antagonist and behavioral, cellular, and/or molecular responses measured and compared to those of a wild-type animal exposed to the same exogenous agent. Consequently, the therapeutic potential of the sensory-related gene therapy regime may be assessed.

Still yet another aspect encompasses a method of generating a cell line or cell lysate using a genetically modified animal comprising an edited chromosomal sequence encoding a sensory-related protein. An additional other aspect encompasses a method of producing purified biological components using a genetically modified cell or animal comprising an edited chromosomal sequence encoding a sensory-related protein. Non-limiting examples of biological components include antibodies, cytokines, signal proteins, enzymes, receptor agonists and receptor antagonists.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The terms “polypeptide” and “protein” are used interchangeably to refer to a polymer of amino acid residues.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a zinc finger nuclease is engineered to recognize and bind, provided sufficient conditions for binding exist.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between regions that share a degree of sequence identity, followed by digestion with single-stranded-specific nuclease(s), and size determination of the digested fragments. Two nucleic acid, or two polypeptide sequences are substantially similar to each other when the sequences exhibit at least about 70%-75%, preferably 80%-82%, more-preferably 85%-90%, even more preferably 92%, still more preferably 95%, and most preferably 98% sequence identity over a defined length of the molecules, as determined using the methods above. As used herein, substantially similar also refers to sequences showing complete identity to a specified DNA or polypeptide sequence. DNA sequences that are substantially similar can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Conditions for hybridization are well-known to those of skill in the art.

Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations. With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. A particular set of hybridization conditions may be selected following standard methods in the art (see, for example, Sambrook, et al., Molecular Cloning: A Laboratory Manual, Second Edition, (1989) Cold Spring Harbor, N.Y.).

### EXAMPLES

The following examples are included to illustrate the invention.

### Example 1

### Genome Editing of TRPM5 Locus

Zinc finger nucleases (ZFNs) that target and cleave the TRPM5 locus of rats may be designed, assembled, and validated using strategies and procedures previously described (see Geurts et al. Science (2009) 325:433). ZFN design may make use of an archive of pre-validated 1-finger and 2-finger modules. The rat TRPM5 gene region was scanned for putative zinc finger binding sites to which existing modules could be fused to generate a pair of 4-, 5-, or 6-finger proteins that would bind a 12-18 by sequence on one strand and a 12-18 by sequence on the other strand, with about 5-6 by between the two binding sites.

Capped, polyadenylated mRNA encoding pairs of ZFNs may be produced using known molecular biology techniques. The mRNA may be transfected into rat cells. Control cells may be injected with mRNA encoding GFP. Active ZFN pairs may be identified by detecting ZFN-induced double strand chromosomal breaks using the Cel-1 nuclease assay. This assay detects alleles of the target locus that deviate from wild type (WT) as a result of non-homologous end joining (NHEJ)-mediated imperfect repair of ZFN-induced DNA double strand breaks. PCR amplification of the targeted region from a pool of ZFN-treated cells generates a mixture of WT and mutant amplicons. Melting and reannealing of this mixture results in mismatches forming between heteroduplexes of the WT and mutant alleles. A DNA “bubble” formed at the site of mismatch is cleaved by the surveyor nuclease Cel-1, and the cleavage products can be resolved by gel electrophoresis. This assay may be used to identify a pair of active ZFNs that edited the TRPM5 locus.

To mediate editing of the TRPM5 gene locus in animals, fertilized rat embryos may be microinjected with mRNA encoding the active pair of ZFNs using standard procedures (e.g., see Geurts et al. (2009) supra). The injected embryos may be either incubated in vitro, or transferred to pseudopregnant female rats to be carried to parturition. The resulting embryos/fetus, or the toe/tail clip of live born animals may be harvested for DNA extraction and analysis. DNA may be isolated using standard procedures. The targeted region of the TRPM5 locus may be PCR amplified using appropriate primers. The amplified DNA may be subcloned into a suitable vector and sequenced using standard methods.

### Example 2

### Genome Editing of ERAL1 in a Model Organism

ZFN-mediated genome editing may be used to study the effects of a “knockout” mutation in nociception-related chromosomal sequence, such as a chromosomal sequence encoding the ERAL1 protein, in a genetically modified model animal and cells derived from the animal. Such a model animal may be a rat. In general, ZFNs that bind to the rat chromosomal sequence encoding the ERAL1 protein associated with a nociception pathway may be used to introduce a deletion or insertion such that the coding region of the ERAL1 gene is disrupted such that a functional ERAL1 protein may not be produced.

Suitable fertilized embryos may be microinjected with capped, polyadenylated mRNA encoding the ZFN essentially as detailed above in Example 1. The frequency of ZFN-induced double strand chromosomal breaks may be determined using the Cel-1 nuclease assay, as detailed above. The sequence of the edited chromosomal sequence may be analyzed as described above. The development of AD symptoms and disorders caused by the ERAL1 “knockout” may be assessed in the genetically modified rat or progeny thereof. Furthermore, molecular analyses of nociception-related pathways may be performed in cells derived from the genetically modified animal comprising a ERAL1 “knockout”.

### Example 3

### Generation of a Humanized Rat Expressing a Mutant Form of Human SCN9A

Missense mutations in SCN9A, a sodium ion channel that is expressed at high levels in nociceptive dorsal root ganglion (DRG) neurons, are associated with erythromelagia, an inherited disorder characterized by symmetrical burning pain of the feet, lower legs, and hands. Three mutations have been characterized in SCN9A: W897X, located in the P-loop of domain 2; 1767X, located in the S2 segment of domain 2; and S459X, located in the linker region between domains 1 and 2, any one of which results in a truncated non-functional protein. ZFN-mediated genome editing may be used to generate a humanized rat wherein the rat SCN9A gene is replaced with a mutant form of the human SCN9A gene comprising the W897X mutation, the I767X mutation, the S459X mutation, or any combination of the three mutations. Such a humanized rat may be used to study the development of the erythromelagia associated with the mutant human SCN9A protein. In addition, the humanized rat may be used to assess the efficacy of potential therapeutic agents targeted at the pathway leading to erythromelagia comprising SCN9A.

The genetically modified rat may be generated using the methods described in Example 1 above. However, to generate the humanized rat, the ZFN mRNA may be co-injected with the human chromosomal sequence encoding the mutant SCN9A protein into the rat embryo. The rat chromosomal sequence may then be replaced by the mutant human sequence by homologous recombination, and a humanized rat expressing a mutant form of the SCN9A protein may be produced.

What is claimed is:

1. A genetically modified animal comprising at least one edited chromosomal sequence encoding a sensory-related protein.

2. The genetically modified animal of claim 1, wherein the edited chromosomal sequence is inactivated, modified, or comprises an integrated sequence.

3. The genetically modified animal of claim 1, wherein the edited chromosomal sequence is inactivated such no functional sensory-related protein is produced.

4. The genetically modified animal of claim 3, wherein inactivated chromosomal sequence comprises no exogenously introduced sequence.

5. The genetically modified animal of claim 3, further comprising at least one chromosomally integrated sequence encoding a functional sensory-related protein.

6. The genetically modified animal of claim 1, wherein the sensory-related protein is chosen from TRPM5, TRPM7, TRPC1, TRPC5, TRPC6, TRPA1, CNR1, CNR2, POMC, CALCA, CRF, PRKACA, PRKACB, PRKAR1A, PRKAR2A, ERAL1, NR2B, LGALS1, TRPV1, SCN9A, OPRM1, OPRD1, OPRK1, and combinations thereof.

7. The genetically modified animal of claim 1, further comprising a conditional knock-out system for conditional expression of the sensory-related protein.

8. The genetically modified animal of claim 1, wherein the edited chromosomal sequence comprises an integrated reporter sequence.

9. The genetically modified animal of claim 1, wherein the animal is heterozygous or homozygous for the at least one edited chromosomal sequence.

10. The genetically modified animal of claim 1, wherein the animal is an embryo, a juvenile, or an adult.

11. The genetically modified animal of claim 1, wherein the animal is chosen from bovine, canine, equine, feline, ovine, porcine, non-human primate, and rodent.

12. The genetically modified animal of claim 1, wherein the animal is rat.

13. The genetically modified animal of claim 4, wherein the animal is rat and the protein is an ortholog of a human sensory-related protein.

14. A non-human embryo comprising at least one RNA molecule encoding a zinc finger nuclease that recognizes a chromosomal sequence encoding a sensory-related protein, and, optionally, at least one donor polynucleotide comprising a sequence encoding the sensory-related protein or an edited sensory-related protein.

15. The non-human embryo of claim 14, wherein the sensory-related protein is chosen from TRPM5, TRPM7, TRPC1, TRPC5, TRPC6, TRPA1, CNR1, CNR2, POMC, CALCA, CRF, PRKACA, PRKACB, PRKAR1A, PRKAR2A, ERAL1, NR2B, LGALS1, TRPV1, SCN9A, OPRM1, OPRD1, OPRK1, and combinations thereof.

16. The non-human embryo of claim 14, wherein the embryo is chosen from bovine, canine, equine, feline, ovine, porcine, non-human primate, and rodent.

17. The non-human embryo of claim 14, wherein the embryo is rat and the protein is an ortholog of a human sensory-related protein.

18. A genetically modified cell, the cell comprising at least one edited chromosomal sequence encoding a sensory-related protein.

19. The genetically modified cell of claim 18, wherein the edited chromosomal sequence is inactivated, modified, or comprises an integrated sequence.

20. The genetically modified cell of claim 18, wherein the edited chromosomal sequence is inactivated such that no functional sensory-related protein is produced.

21. The genetically modified cell of claim 20, further comprising at least one chromosomally integrated sequence encoding a functional sensory-related protein.

22. The genetically modified cell of claim 18, wherein the sensory-related protein is chosen from TRPM5, TRPM7, TRPC1, TRPC5, TRPC6, TRPA1, CNR1, CNR2, POMC, CALCA, CRF, PRKACA, PRKACB, PRKAR1A, PRKAR2A, ERAL1, NR2B, LGALS1, TRPV1, SCN9A, OPRM1, OPRD1, OPRK1, and combinations thereof.

23. The genetically modified cell of claim 18, wherein the cell is heterozygous or homozygous for the at least one edited chromosomal sequence.

24. The genetically modified cell of claim 18, wherein the cell is of bovine, canine, equine, feline, human, ovine, porcine, non-human primate, or rodent origin.

25. The genetically modified cell of claim 18, wherein the cell is of rat origin and the protein is an ortholog of a human sensory-related protein.

26. A method for assessing an effect of an agent in an animal, the method comprising:
a) administering the agent to a genetically modified animal comprising at least one edited chromosomal sequence encoding a sensory-related protein;
b) obtaining a parameter from the genetically modified animal, wherein the parameter is chosen from any one or more of:
i. rate of elimination of the agent or at least one agent metabolite;
ii. circulatory levels of the agent or the at least one agent metabolite;
iii. bioavailability of the agent or the at least one agent metabolite;
iv. rate of metabolism of the agent or the at least one agent metabolite;
v. rate of clearance of the agent or the at least one agent metabolite;
vi. toxicity of the agent or the at least one agent metabolite;
vii. disposition of the agent or the at least one agent metabolite;
viii. extrahepatic contribution to the rate of metabolism or the rate of clearance of the agent or the at least one agent metabolite; and
ix. ability of the agent to modify an incidence or indication of a sensory disorder in the genetically modified animal, wherein the sensory disorder is chosen from a nociception disorder, a taste disorder, or any combination thereof; and
c) comparing the parameter obtained from the genetically modified animal to the selected obtained from a wild-type animal administered the same agent.

27. The method of claim 26, wherein the agent is a pharmaceutically active ingredient, a drug, a toxin, or a chemical.

28. The method of claim 26, wherein the at least one edited chromosomal sequence is inactivated such that no functional sensory-related protein is produced, and wherein the genetically modified animal further comprises at least one chromosomally integrated sequence encoding a functional ortholog of the sensory-related protein.

29. The method of claim 26, wherein the sensory-related protein is chosen from TRPM5, TRPM7, TRPC1, TRPC5, TRPC6, TRPA1, CNR1, CNR2, POMC, CALCA, CRF, PRKACA, PRKACB, PRKAR1A, PRKAR2A, ERAL1, NR2B, LGALS1, TRPV1, SCN9A, OPRM1, OPRD1, OPRK1, and combinations thereof.

30. The method of claim 26, wherein the animal is a rat of a strain chosen from Dahl Salt-Sensitive, Fischer 344, Lewis, Long Evans Hooded, Sprague-Dawley, and Wistar.

31. A method for assessing an indication of a sensory disorder chosen from a nociception disorder, a taste disorder, and combinations thereof in an animal model comprising a genetically modified animal comprising at least one edited chromosomal sequence encoding a sensory-related protein, the method comprising comparing a selected parameter obtained from the animal model to the selected parameter obtained from a wild-type animal, wherein the selected parameter is chosen from:
a) spontaneous behaviors;
b) performance during behavioral testing;
c) physiological anomalies;
d) abnormalities in tissues or cells;
e) biochemical function; and
f) molecular structures.

32. The method of claim 31, wherein the at least one indication of the sensory disorder occurs spontaneously in the animal model.

33. The method of claim 31, wherein the at least one indication of the sensory disorder is promoted by exposure to an exogenous agent chosen from an nocioception-stimulating agent, a taste-stimulating agent, a sensory-related protein, a sensory-related agonist, and a sensory-related antagonist.

34. A method for assessing at least one side effect of a therapeutic compound comprising administering the therapeutic compound to an animal model, wherein the animal model is chosen from a genetically modified animal and a wild-type animal, wherein the genetically modified animal comprises at least one edited chromosomal sequence encoding a sensory-related protein, and assessing at least one or more behaviors chosen from learning, memory, anxiety, depression, addiction, sensory-motor function, taste preference, and odor preference.

35. The method of claim 34, wherein the therapeutic compound is chosen from a novel therapeutic compound and a novel combination of known therapeutic agents.

36. The method of claim 34, wherein the animal model further comprises a wild-type animal.

37. The method of claim 34, wherein the treatment with the therapeutic compound is self-administered.

38. The method of claim 34, wherein the treatment with the therapeutic compound is administered by incorporating the therapeutic compound in an amount of water, food, or supplemental material provided to the animal model.
